(19) United States
(12) Patent Application Publication
(10) Pub. No.: US 2022/0226305 A1
(43) Pub. Date: Jul. 21, 2022

(54) METHOD AND COMBINATION FOR TREATING VIRAL INFECTION AND LONG HAULER SYNDROME

(71) Applicant: Sabine Hazan, Ventura, CA (US)

(72) Inventor: Sabine Hazan, Ventura, CA (US)

(21) Appl. No.: 17/714,760

(22) Filed: Apr. 6, 2022

Related U.S. Application Data
(63) Continuation-in-part of application No. 17/406,774, filed on Aug. 19, 2021.
Application No. 17/406,774 is a continuation-in-part of application No. 17/200,585, filed on Mar. 12, 2021.
Application No. 17/200,585 is a continuation-in-part of application No. 17/026,051, filed on Sep. 18, 2020, now Pat. No. 11,253,534.
(60) Provisional application No. 62/993,345, filed on Mar. 23, 2020.
(60) Provisional application No. 63/022,371, filed on May 8, 2020.
(60) Provisional application No. 63/002,494, filed on Mar. 31, 2020.
(60) Provisional application No. 63/022,368, filed on May 8, 2020.
(60) Provisional application No. 63/001,161, filed on Mar. 27, 2020.
(60) Provisional application No. 62/993,345, filed on Mar. 23, 2020.
(60) Provisional application No. 63/022,371, filed on May 8, 2020.
(60) Provisional application No. 63/002,494, filed on Mar. 31, 2020.
(60) Provisional application No. 63/022,368, filed on May 8, 2020.
(60) Provisional application No. 63/001,161, filed on Mar. 27, 2020.

(51) Int. Cl. A61K 31/4706; A61K 31/375; A61K 31/593; A61K 31/65; A61K 31/7048; A61K 31/7052; A61K 33/30; A61P 31/14; A61K 45/06

(52) U.S. Cl. CPC A61K 31/4706; A61K 31/375; A61K 31/593; A61K 31/65; A61K 45/06; A61K 31/7052; A61K 33/30; A61P 31/14; A61K 31/7048

(57) ABSTRACT

A method of treating an individual having a viral infection or long hauler syndrome, the method comprising the steps of: providing an individual having a viral infection; and administering eight antivirals to the individual, wherein the antivirals comprise: hydroxychloroquine, azithromycin or doxycycline, ivermectin, oseltamivir phosphate, vitamin c, vitamin d, zinc, and bifidobacterial probiotics. Optionally, the method can also comprise administering famotidine, aspirin, cyproheptadine, a budesonide inhaler, and an iodine nasal spray. A combination of two or more antivirals for treating a viral infection or long hauler syndrome, the combination comprising at least two of the following: hydroxychloroquine, azithromycin or doxycycline, ivermectin, oseltamivir phosphate, famotidine, vitamin c, vitamin d, zinc, aspirin, bifidobacterial probiotics, cyproheptadine, budesonide inhaler, and iodine nasal spray.

### CROSS REFERENCE TO RELATED APPLICATIONS

This application is a continuation-in-part of U.S. patent application Ser. No. 17/406,774, titled “Methods of Preventing and Treating COVID-19 Infection with Probiotics,” filed Aug. 19, 2021, which is a continuation-in-part of U.S. patent application Ser. No. 17/200,585, titled “Methods of Preventing and Treating COVID-19 Infection with Probiotics,” filed Mar. 12, 2021, which claims priority to U.S. patent application Ser. No. 17/026,051, now U.S. Pat. No. 11,253,534, titled “Method of Preventing and Treating COVID-19 Infection, filed Sep. 18, 2020, which claims priority to U.S. Provisional Patent Application No. 62/993,345, titled “Method of Treating and Preventing COVID-19 Infection,” filed Mar. 23, 2020, U.S. Provisional Patent Application No. 63/022,371, titled “Method of Treating and Preventing COVID-19 Infection,” filed May 8, 2020, U.S. Provisional Patent Application No. 63/002,494, titled “Method of Using Vitamin C, Vitamin D, Zinc, and Optionally Hydroxychloroquine, to Prevent COVID-19 Infection,” filed Mar. 31, 2020, U.S. Provisional Patent Application No. 63/022,368, titled “Method of Using Vitamin C, Vitamin D, Zinc, and Optionally Hydroxychloroquine, to Prevent COVID-19 Infection,” filed May 8, 2020, and U.S. Provisional Patent Application No. 63/001,161, titled “Method of Using Aerosolized Hydroxychloroquine, Vitamin C, and Zinc to Treat Covid-19 Infection,” filed Mar. 27, 2020, and is continuation-in-part of U.S. patent application Ser. No. 17/350,520, titled “Method of Preventing and Treating COVID-19 Infection,” filed Jun. 17, 2021, which is a continuation application of U.S. patent application Ser. No. 17/126,715, now U.S. Pat. No. 11,166,971, titled “Method of Preventing and Treating COVID-19 Infection,” filed Dec. 18, 2020, which is a divisional application of U.S. patent application Ser. No. 17/026,051, titled “Method of Preventing and Treating COVID-19 Infection, filed Sep. 18, 2020, which claims priority to U.S. Provisional Patent Application No. 62/993,345, titled “Method of Treating and Preventing COVID-19 Infection,” filed Mar. 23, 2020, U.S. Provisional Patent Application No. 63/022,371, titled “Method of Treating and Preventing COVID-19 Infection,” filed May 8, 2020, U.S. Provisional Patent Application No. 63/002,494, titled “Method of Using Vitamin C, Vitamin D, Zinc, and Optionally Hydroxychloroquine, to Prevent COVID-19 Infection,” filed Mar. 31, 2020, U.S. Provisional Patent Application No. 63/022,368, titled “Method of Using Vitamin C, Vitamin D, Zinc, and Optionally Hydroxychloroquine, to Prevent COVID-19 Infection,” filed May 8, 2020, and U.S. Provisional Patent Application No. 63/001,161, titled “Method of Using Aerosolized Hydroxychloroquine, Vitamin C, and Zinc to Treat Covid-19 Infection,” filed Mar. 27, 2020, the contents of which are incorporated by reference in their entirety.

### BACKGROUND

Viral infections are a constant and recurring issue for human health. With the emergence of COVID-19, viral infections have become a focus of the health industry.

It is known that single anti-viral agents work poorly when used alone in other chronic viral infections such as Hepatitis C or HIV infection. Therefore, the greater the number of anti-viral agents used in combination, the greater the cure rate.

Thus, there is a significant unmet need for treating viral infections and long hauler syndrome.

### SUMMARY

The present invention addresses this need. The invention herein is directed to my method of treating viral infection and long hauler syndrome. The method of treatment comprises the steps of: providing an individual having a viral infection; and administering at least eight antimicrobials to the individual.

The antimicrobials comprise: administering hydroxychloroquine in a daily dosage range of 20 mg to 2,000 mg; administering azithromycin or doxycycline in a daily dosage range of 250 mg to 500 mg; administering ivermectin in a daily dosage range of 3 mg to 200 mg; administering vitamin C in a daily dosage range of 1,000 mg to 20,000 mg; administering vitamin D in a daily dosage range of 1,000 IU to 100,000 IU; administering zinc in a daily dosage range of 25 mg to 100 mg; and administering bifidobacterial probiotics in a daily dosage range of 1 billion to 100,000 billion bifidobacterial.

Optionally, the method can further comprise administering famotidine in a daily dosage range of 20 mg to 80 mg; administering oseltamivir phosphate in a daily dosage range of 30 mg to 75 mg; administering aspirin in a daily dosage range of 81 mg to 500 mg; administering cyproheptadine in a daily dosage range of 4 mg to 8 mg; administering a budesonide inhaler in daily dosage range of one to two puffs; and administering an iodine nasal spray in a daily dosage range of one spray in one nostril to two sprays in both nostrils.

Optionally, 400 mg of hydroxychloroquine can be administered, 250 mg of azithromycin or doxycycline can be administered, 36 mg of ivermectin can be administered, and 8 mg of cyproheptadine can be administered.

The method can be administered for at least one day, at least two days, or fourteen days.

The method can further comprise monitoring the individual's condition over a pre-determined period of time to determine whether the individual no longer has the viral infection.

The probiotic can be administered anally, orally, or topically, and if the probiotic is administered anally, it can be in the form of a fecal transplant, colonoscopy, suppository, enema, or topical application.

A combination of two or more antivirals for treating a viral infection, the combination comprising at least two of the following: hydroxychloroquine, azithromycin or doxycycline, ivermectin, oseltamivir phosphate, famotidine, vitamin c, vitamin d, zinc, aspirin, bifidobacterial probiotics, cyproheptadine, budesonide inhaler, and iodine nasal spray.

Preferably, the combination comprises at least hydroxychloroquine, azithromycin or doxycycline, ivermectin, oseltamivir phosphate, vitamin c, vitamin d, zinc, and bifidobacterial probiotics.

Optionally, combination comprises: 20 mg to 2,000 mg of hydroxychloroquine, 250 mg to 500 mg of azithromycin or doxycycline, 3 mg to 200 mg of ivermectin, 30 mg to 75 mg of oseltamivir phosphate, 1,000 mg to 20,000 mg of vitamin C, 1,000 IU to 100,000 IU of vitamin D, 25 mg to 100 mg of zinc, and 1 billion to 100,000 billion bifidobacterial in the form of a probiotic.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1 is a flow chart depicting the steps of a method of preventing infection of an individual with COVID-19;

FIG. 2 is a flow chart depicting the steps of a first method of treating an individual infected with COVID-19;

FIG. 3A a graphical representation of whole genome alignment of SARS-CoV-2 in patient 1 of Example 6;

FIG. 3B a graphical representation of whole genome alignment of SARS-CoV-2 in patient 3 of Example 6;

FIG. 3C a graphical representation of whole genome alignment of SARS-CoV-2 in patient 4 of Example 6;

FIG. 3D a graphical representation of whole genome alignment of SARS-CoV-2 in patient 6 of Example 6;

FIG. 3E a graphical representation of whole genome alignment of SARS-CoV-2 in patient 8 of Example 6;

FIG. 3F a graphical representation of whole genome alignment of SARS-CoV-2 in patient 10 of Example 6;

FIG. 3G a graphical representation of whole genome alignment of SARS-CoV-2 in patient 11 of Example 6;

FIG. 3H a graphical representation of whole genome alignment of SARS-CoV-2 in patient 12 of Example 6;

FIG. 4A is a graph showing the Alpha diversity of SARS-CoV-2 positive patients and exposed healthy controls using a Shannon index [P=0.026];

FIG. 4B is a graph showing the Alpha diversity index of SARS-CoV-2 positive patients and exposed healthy controls using a Simpson index [P=0.043];

FIG. 5 is a graph showing the relative abundance of Bifidobacteria for each subject, grouped by COVID-19 severity;

FIG. 6 is a diagram showing a proposed mechanism for cytokine storm and immune hyper-response in SARS-CoV-2 positive patients; and

FIG. 7 is a flow chart depicting the steps of a second method of treating an individual with a viral infection.

### DETAILED DESCRIPTION

As used herein, the following terms and variations thereof have the meanings given below, unless a different meaning is clearly intended by the context in which such term is used.

The terms “a,” “an,” and “the” and similar referents used herein are to be construed to cover both the singular and the plural unless their usage in context indicates otherwise.

As used in this disclosure, the term “comprise” and variations of the term, such as “comprising” and “comprises,” are not intended to exclude other additives, components, integers ingredients or steps.

Referring now to the drawings, wherein like reference numerals designate identical or corresponding features throughout the several views. Further, described herein are certain non-limiting embodiments of my pipeline filter assembly for pool filtering and maintenance.

The following discussion describes in detail multiple embodiments of the invention with several variations of those embodiments. This discussion should not be construed, however, as limiting the invention to those particular embodiments. Practitioners skilled in the art will recognize numerous other embodiments as well.

In a first embodiment, the present invention is directed to a method of preventing COVID-19 infection in an individual. The method involves administration of chloroquine or hydroxychloroquine, Vitamin C, Vitamin D, and Zinc. Both methods are discussed in greater detail below.

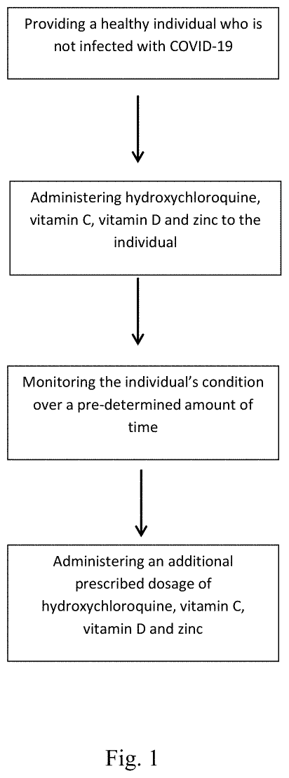

Referring now to FIG. 1, there is shown the method of prevention. The method of prevention comprises administering four different antimicrobials. The four antimicrobials comprise: chloroquine or hydroxychloroquine, vitamin C, vitamin D, and zinc.

Day 1 the individual takes the following, ideal, regimen outlined in Table 1:

TABLE 1

| Drug | AM Dose | PM Dose |
|---|---|---|
| Hydroxychloroquine | 200 mg | 200 mg |
| Vitamin C | 3000 mg | — |
| Vitamin D | 3000 mg | — |
| Zinc | 50 mg | — |

Optionally, the method of prevention can comprise administering on day 2: 3,000 mg of vitamin C; 3,000 mg of vitamin D; and 50 mg of zinc.

Hydroxychloroquine is administered only on day 1. The half-life of hydroxychloroquine is up to 32 days, thus treatment with this drug for one day should be sufficient. However, should the need to prevent the infection or disease last longer than 32 days, repeat dosing can be considered. Accordingly, if necessary, the cycle of day 1 followed by day 2 can be repeated weekly, 2^{nd}-weekly, every 3 weeks, 4 weeks, 5 weeks, 6 weeks, 7 weeks, or 8 weeks.

Vitamin C is administered at 3,000 mg per day ongoing. This 3,000 mg can be broken up into two 1500 mg doses, one taken in the morning and one taken at night.

Vitamin D is administered at 3,000 mg per day ongoing. This 3,000 mg can be broken up into two 1500 mg doses, one taken in the morning and one taken at night.

Zinc is administered at 50 mg per day ongoing. This 50 mg can be broken up into two 25 mg doses, one taken in the morning and one taken at night.

Chloroquine or hydroxychloroquine can be administered in a daily dosage range of 20 mg to 2,000 mg. The above amounts recited in the tables are not limiting.

Vitamin C can be administered in a daily dosage range of 250 mg to 10,000 mg. The above amounts recited in the tables are not limiting.

Vitamin D can be administered in a daily dosage rage of 1,000 mg to 100,000 mg. The above amounts recited in the tables are not limiting.

Zinc can be administered in a daily dosage of 5 mg to 100 mg. The amount of Zinc can be reduced to 25 mg per day if gastrointestinal upset occurs.

Zinc, Vitamin C and D help in numerous aspects of viral protection through cellular metabolism, including catalytic activity of enzymes, and play roles in immune function, protein synthesis.

The chloroquine or hydroxychloroquine can come in various forms: as a pill, liquid solution, lozenges, topical treatment such as a cream or oil, or any other means of delivery. Hydroxychloroquine prevents cytokine release, and cytokine release is what causes anaphylactic flush. Optionally, the hydroxychloroquine is sprayed directly on the users tongue.

In the method of prevention described above, all, or any combination of, the four antimicrobials disclosed above can be administered in the form of a single small atomizer. The patient sprays the atomizer towards the back of their throat. The spray is administered at least once a day, but preferably twice a day when coughing starts. Use of the atomizer continues as directed by the supervising physician.

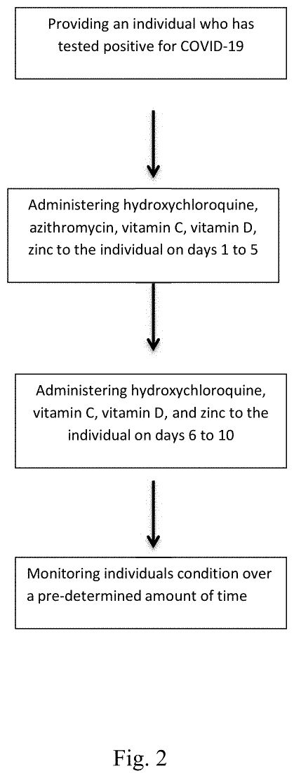

In a second embodiment, the present invention is directed to a method of treating an individual with infection or disease with five different antimicrobials. The five antimicrobials comprise: hydroxychloroquine, azithromycin, vitamin C, vitamin D and zinc. Referring now to FIG. 2, there is shown the method of treatment.

Day 1 following positive test (isolation), the individual takes the following regimen outlined in Table 2:

TABLE 2

| | | |
|---|---|---|
| Drug | AM Dose | PM Dose |

| | | | |
|---|---|---|---|
| Hydroxychloroquine | 200 | mg | 200 mg |
| Azithromycin | 500 | mg | — |
| Vitamin C | 3000 | mg | — |
| Vitamin D | 3000 | mg | — |
| Zinc | 50 | mg | — |

On Day 2-Day 5 the individual takes the following regimen outlined in Table 3:

TABLE 3

| | | |
|---|---|---|
| Drug | AM Dose | PM Dose |

| | | | |
|---|---|---|---|
| Hydroxychloroquine | 200 | mg | 200 mg |
| Azithromycin | 250 | mg | — |
| Vitamin C | 3000 | mg | — |
| Vitamin D | 3000 | mg | — |
| Zinc | 50 | mg | — |

On Day 6-Day 10, the individual takes the following regimen outlined in Table 4:

TABLE 4

| Drug | AM Dose | PM Dose |
|---|---|---|
| Hydroxychloroquine | 200 mg | 200 mg |
| Vitamin C | 3000 mg | — |
| Vitamin D | 3000 mg | — |
| Zinc | 50 mg | — |

Hydroxychloroquine is administered daily, at 200 mg twice daily for days 1-10.

Azithromycin is administered daily, at 500 mg on day 1, and 250 mg days 2-5. Optionally, azithromycin can be administered at 250 mg per day for 5-10 days with a loading dose of 500 mg×day 1 or simply 250 mg on day 1. Azithromycin may be substituted with doxycycline at a daily dosage range of 25 mg to 800 mg, for those unable to take azithromycin.

Vitamin C is administered at 3,000 mg per day ongoing. This 3,000 mg can be broken up into two 1500 mg doses, one taken in the morning and one taken at night.

Vitamin D is administered at 3,000 mg per day ongoing. This 3,000 mg can be broken up into two 1500 mg doses, one taken in the morning and one taken at night.

Zinc is administered at 50 mg per day ongoing. This 50 mg can be broken up into two 25 mg doses, one taken in the morning and one taken at night.

Chloroquine or hydroxychloroquine can be administered in a daily dosage range of 20 mg to 2,000 mg. The above amounts recited in the tables are not limiting.

Vitamin C can be administered in a daily dosage range of 250 mg to 10,000 mg. The above amounts recited in the tables are not limiting.

Vitamin D can be administered in a daily dosage rage of 1,000 mg to 100,000 mg. The above amounts recited in the tables are not limiting.

Zinc can be administered in a daily dosage of 5 mg to 100 mg. The amount of Zinc can be reduced to 25 mg per day if gastrointestinal upset occurs.

Concurrently with the above treatment, the individual is self-quarantined per CDC recommendations.

Optionally, for the method of treatment noted above, as the dosages of the hydroxychloroquine, vitamin C, vitamin D and zinc remain the same throughout treatment, all four of those antimicrobials can be administered in the form of a single small atomizer. The patient sprays the atomizer towards the back of their throat. The spray is administered at least once a day, but preferably twice a day when coughing starts. Use of the atomizer continues as directed by the supervising physician.

In an additional embodiment, the present invention is directed to a method of treatment of viral infections and long hauler syndrome. Long hauler syndrome is when a individual has had a viral infection, has not fully recovered, and continues to have symptoms over a long period of time, such as weeks or months, or they continue to relapse with symptoms.

The method comprises administering one or more of the following compounds/components/antivirals for at least one (1) day, and up to fourteen (14) days: hydroxychloroquine, azithromycin or doxycycline, ivermectin, oseltamivir phosphate (Tamiflu™), famotidine (Pepcid™), vitamin c, vitamin d, zinc, aspirin, bifidobacterial probiotics, cyproheptadine, budesonide inhaler, and iodine nasal spray.

Preferably, at least hydroxychloroquine, azithromycin or doxycycline, ivermectin, oseltamivir phosphate (Tamiflu™), vitamin c, vitamin d, zinc, and bifidobacterial probiotics are administered.

Hydroxychloroquine or chloroquine can be administered as follows: orally, daily in a dosage range of 20 mg to 2,000 mg (including any amounts or ranges in between). Preferably, 200 mg is administered twice daily for 1 to 14 days.

Azithromycin or doxycycline can be administered as follows: administered daily, at 500 mg on day 1, and 250 mg days 2-14. Azithromycin may be substituted with doxycycline at a daily dosage range of 25 mg to 800 mg (including any amount or range in between), for those unable to take azithromycin.

Ivermectin can be administered as follows: 3 mg to 200 mg (including any amount or range in between), orally, daily, for 1 to 14 days; preferably, 36 mg, orally, twice a day, for 1 to 14 days.

Oseltamivir phosphate can be administered twice a day orally in the form of 30 mg, 45 mg, or 75 mg capsules or as an oral suspension made from 360 mg oseltamivir base supplied as powder (constituted to a final concentration of 6 mg/mL), for 1 to 14 days. Additional dosage options are as follows:
Adults and adolescents (13 years and older): 75 mg twice daily for 5 days
Pediatric patients 1 to 12 years of age: Based on weight twice daily for 5 days
Pediatric patients 2 weeks to less than 1 year of age: 3 mg/kg twice daily for 5 days
Renally impaired adult patients (creatinine clearance >30-60 mL/min): Reduce to 30 mg twice daily for 5 days
Renally impaired adult patients (creatinine clearance >10-30 mL/min): Reduce to 30 mg once daily for 5 days
ESRD patients on hemodialysis: Reduce to 30 mg immediately and then 30 mg after every hemodialysis cycle. Treatment duration not to exceed 5 days
ESRD patients on CAPD: Reduce to a single 30 mg dose immediately

Famotidine can be administered as follows: 20 mg to 80 mg (including any amounts or ranges in between), orally, three times a day, for 1 to 14 days.

Vitamin C can be administered as follows: 1000 mg to 20,000 mg (including any amounts or ranges in between), orally, daily for 1 to 14 days.

Vitamin D can be administered as follows: 1,000 IU to 100,000 IU (including any amounts or ranges in between), orally, daily for 1 to 14 days.

Zinc can be administered as follows: any form or type of zinc: 50 mg to 100 mg (including any amounts or ranges in between), orally, daily for 1 to 14 days

Aspirin can be administered as follows: 81 mg to 325 mg to 500 mg (including any amounts or ranges in between) orally, daily, for 1 to 14 days.

Bifidobacteria probiotics can be administered as follows: one or more probiotics containing 1 billion to 100,000 billion bifidobacteria (including but not limited to Bifidobacteriales, Bifidobacteriacae) administered orally in form of a pill, lozenge, liquid tincture, spray, drink, chewable tablet, or food stuff such as yogurt, or rectally, in the form of a fecal transplant, suppository, or cream, daily, for 1 to 14 days. In all instances, amount of probiotic, and the amount or dosage of the bacteria contained within the probiotic can vary from patient to patient, depending on the microbiome needs. The probiotics can be administered for as many days as the supervising physician deems necessary.

Cyproheptadine can be administered as follows: 4 mg to 8 mg (including any amount or range in between) orally, daily, for 1 to 14 days; preferably, 4 mg, orally, twice a day, for 1 to 14 days.

Budesonide inhaler can be administered as follows: For inhalation dosage form (powder inhaler): Adults and children 6 years of age and older—At first, one or two puffs two times a day. For inhalation dosage form (suspension in a nebulizer): Children 12 months to 8 years of age—0.5 to 1 milligram (mg) in a nebulizer once a day, or divided and given twice a day. Each container of liquid has one dose and a new container is used for each dose. Regardless of form, the budesonide is administered daily, for 1 to 14 days.

Iodine nasal spray can be administered as follows: daily, one or more squirts in one or both nostrils, for 1 to 14 days.

For all protocols provided in this application, vitamin C dosage can range from 250 mg to 10,000 mg per day (including any amounts or ranges in between), vitamin D dosage can range from 1000 IU (mg) to 100,000 IU (mg) per day (including any amounts or ranges in between), zinc (which can be any form or type of zinc) dosage can range from 5 mg to 100 mg per day (including any amounts or ranges in between), and hydroxychloroquine/chloroquine dosage can range from 50 mg to 2,000 mg per day (including any amounts or ranges in between) for a treatment period of 1 to 14 days treatment (including any range in between). Optionally, hydroxychloroquine can be administered once as single dose. For all protocols provided in this application, when a dosage range is provided, any dosage amount that is included in that range can be administered. Accordingly, the invention is not limited to the dosage ranges disclosed and includes all dosage amounts contained in those ranges.

Optionally, the above protocols can include selenium, copper and other vitamins that are deemed acceptable supplements for vitamin C, vitamin D or zinc or to counteract the negative depletion of certain vitamins, which is why copper or selenium are typically used.

The above protocols can be used to treat all viruses, including but not limited to, covid-19, other flu and various respiratory viruses, including more benign coronaviruses and rhinoviruses, Ebola,

The above protocols can also be used to treat Autism, Parkinson's, Alzheimer's and other neurological diseases.

In an additional embodiment, the present invention is directed to the combination of one or more antivirals for treating a viral infection. The combination can comprise one or more of the following: hydroxychloroquine, azithromycin or doxycycline, ivermectin, oseltamivir phosphate (Tamiflu™), famotidine (Pepcid™), vitamin c, vitamin d, zinc, aspirin, bifidobacterial probiotics, cyproheptadine, budesonide inhaler, and iodine nasal spray, in the amounts/ranges noted above.

### EXAMPLES

### Example 1: Hydroxychloroquine, Vitamin C, Vitamin D, and Zinc for Prevention of COVID-19 Infection

Objective: To determine whether treatment with hydroxychloroquine, vitamin C, vitamin D, and zinc in combination will prevent infection with COVID-19 and to assess the safety and tolerability of hydroxychloroquine, vitamin C, vitamin D, and zinc in healthy, high-risk individuals without hypertension, and no evidence of COVID-19 infection.

Procedure: Day 1: Prescription of study drugs and home health monitoring equipment. The study drugs are hydroxychloroquine 200 mg twice a day for 1 day only, vitamin C 3000 mg per day ongoing, vitamin D 3000 mg per day ongoing, and zinc 50 mg per day ongoing per day. The home health monitoring equipment is an electrocardiogram (EKG) which synchs up with a smartphone

Days 2-7: Patient collects the EKG once during this week using the home health equipment

Weeks 1-23: Patient provides an assessment of any COVID-19 symptoms and continues to collect EKG weekly throughout the remainder of the trial

Week 24: Patient undergoes confirmatory COVID-19 testing which consists of nasopharyngeal (NP) and oropharyngeal (OP) swabs collected according to CDC (Center for Disease Control) protocol. The swabs consist of synthetic fiber swabs with plastic shafts. NP swabs are collected by insertion of a swab into the nostril parallel to the palate. The swab is left in place a few seconds to allow it to absorb secretions. OP swabs are inserted into the oropharynx parallel to the palate, avoiding the tongue. The swab is left in place a few seconds to allow it to absorb secretions. The swabs are then immediately placed in sterile tubes with 2-3 mL of viral transport media. The tubes are placed in biohazard bags then boxes and couriered to the local Public Health Lab.

Table 5 provides a schedule of events for Example 1.

TABLE 5

| | Screening | | Weeks | Week |
|---|---|---|---|---|
| Assessment | (Day 1) | Day 3 | 1-23 | 24 |
| Informed consent and | X | | | |
| demographics | | | | |
| Review of prior and | X | | | |
| concomitant medications | | | | |
| EKG at home | | X | X | |
| Prescription of | X | | | |
| Hydroxychloroquine, | | | | |
| vitamin C, vitamin D, | | | | |
| and zinc | | | | |
| Provision of home health | X | | | |
| EKG and pulse oximeter | | | | |
| Update list of prior and | X | | X | X |
| concomitant medications | | | | |
| Ask about any adverse | | X | X | X |
| events and any serious | | | | |
| adverse events | | | | |
| Evaluation of COVID-19 | | | X | X |
| symptoms | | | | |
| COVID-19 testing | | | | X |
| Blood draw for future | | | | X |
| testing | | | | |
| Vitals in-clinic | | | | X |
| Physical exam | | | | X |

Regarding Table 5: Vitals at home to include EKG and O2Sat, future testing will require separate informed consent and could possibly include antibody or cytokine testing, and vitals in-clinic to include height, weight, blood pressure (following 5 minutes sitting) pulse, respiratory rate, temperature, and oxygen saturation.

### Example 2: Randomized, Double-Blind, Placebo-Controlled Phase IIA Study of Hydroxychloroquine, Vitamin C, Vitamin D, and Zinc for the Prevention of COVID-19 Infection

Objectives: Prevention of COVID-19, Lack of COVID-19 symptoms, and assessment of safety and tolerability

Procedure: Screening Period (Days −5 to −1): Prescription of home health monitoring equipment that includes a thermometer, a pregnancy test if applicable, and a daily diary. There are two groups being studied: Arm 1 and Arm 2.

Arm 1 is prescribed the following antimicrobials: hydroxychloroquine 200 mg twice a day for 1 day only, vitamin C 3000 IU per day for 12 weeks, vitamin D 3000 IU per day for 12 weeks, and zinc 50 mg per day for 12 weeks. The zinc can be reduced to 25 mg if GI upset occurs.

Arm 2 is prescribed the following antimicrobials: Placebo twice a day for 1 day only, vitamin C 3000 IU per day for 12 weeks, vitamin D 3000 IU per day for 12 weeks, and zinc 50 mg per day for 12 weeks. The zinc can be reduced to 25 mg if GI upset occurs.

Day 1: Patient is called to teach them how to use the diary in the EDC, discuss the medication regimen, and answer any questions they may have. Patient takes pregnancy test if applicable, collects a temperature reading, completes their diary, and takes the prescribed treatment regimen. Table 6 outlines the prescribed treatment regimen for Day 1.

TABLE 6

| | | |
|---|---|---|
| Drug | AM Dose | PM Dose |

| | | | |
|---|---|---|---|
| Hydroxychloroquine (or placebo) | 200 | mg | 200 mg |
| Vitamin C | 3000 | mg | — |
| Vitamin D | 3000 | IU | — |
| Zinc | 50 | mg | — |

Day 2: Patient collects a temperature reading, completes their diary, and is called on the phone for assessment of any adverse events or serious adverse events, assessment of any COVID-19 symptoms, the list of prior and concomitant medications is updated, any questions the patient has are answered, and the patient takes their prescribed treatment regimen. Table 7 outlines the prescribed treatment regimen for Day 2.

TABLE 7

| Drug | AM Dose | PM Dose |
|---|---|---|
| Vitamin C | 3000 mg | — |
| Vitamin D | 3000 IU | — |
| Zinc | 50 mg | — |

Days 3-10: Patient collects a temperature reading, completes their diary, and takes their prescribed treatment regimen. Table 8 outlines the prescribed treatment regimen for Days 3-10.

TABLE 8

| Drug | AM Dose | PM Dose |
|---|---|---|
| Vitamin C | 3000 mg | — |
| Vitamin D | 3000 IU | — |
| Zinc | 50 mg | — |

Day 14: The patient is called for instruction on how to collect a nasal swab and package for shipping, assessment COVID-19 symptoms, updating their list of prior and concomitant medications, and answering any questions they may have.

Weeks 3-11: The patient is called weekly for assessment of any adverse events or serious adverse events, assessment of any COVID-19 symptoms, updating their list of prior and concomitant medications, and answering any questions they may have. The patient takes weekly temperature readings, completes their diary, and takes the prescribed treatment regimen. During Week 4 the patient is reminded to collect a nasal swab. The prescribed treatment regimen for Weeks 3-11 is shown in Table 9.

TABLE 9

| Drug | AM Dose | PM Dose |
|---|---|---|
| Vitamin C | 3000 mg | — |
| Vitamin D | 3000 IU | — |
| Zinc | 50 mg | — |

Week 12: The patient presents to the clinic for evaluation that includes assessment for adverse events and serious adverse events, updating their list of prior and concomitant medications, a physical exam, nasal swab collection and COVID-19 sample collection.

Samples for COVID-19 testing are collected using synthetic swabs with plastic shafts. Nasal swabs are collected and immediately placed into a sterile vial with 2-3 mL of viral transport media. The vials are placed into biohazard bags, boxed up, the box sterilized, and picked up for shipment to the central laboratory. Samples are tested by RT-PCR.

Table 10 presents the schedule of events for Example 2.

TABLE 10

| | | | | Day | | Weeks | | | |
|---|---|---|---|---|---|---|---|---|---|
| Assessment | Screening | Day 1 | Day 2 | 3-10 | Day 14 | 3-12 | Month 1 | Month 2 | Month 3 |
| Informed Consent | X | | | | | | | | |
| & Demographics | | | | | | | | | |
| Initial Review of | X | | | | | | | | |
| Prior and | | | | | | | | | |
| Concomitant | | | | | | | | | |
| Medications | | | | | | | | | |
| Medical Records | X | | | | | | | | |
| review, including | | | | | | | | | |
| baseline EKG | | | | | | | | | |
| Randomization | X | | | | | | | | |
| Prescription of | X | | | | | | | | |
| Hydroxychloroquine | | | | | | | | | |
| or placebo, vitamin C, | | | | | | | | | |
| vitamin D, and zinc | | | | | | | | | |
| Provision of daily | X | | | | | | | | |
| diary, thermometer | | | | | | | | | |
| and pregnancy test | | | | | | | | | |
| Pregnancy Test if | | X | | | | | | | |
| applicable | | | | | | | | | |
| Temperature at home | | X | X | X | X | X | X | X | X |
| Complete daily diary | | X | X | X | X | X | X | X | X |
| Hydroxychloroquine | | X | | | | | | | |
| 200 mg | | | | | | | | | |
| Vitamin C 3000 mg | | X | X | X | X | X | X | X | X |
| Vitamin D 3000 IU | | X | X | X | X | X | X | X | X |
| Zinc 50 mg^{a} | | X | X | X | X | X | X | X | X |
| Phone/video call | X | X | X | | X | X | X | X | |
| to patient^{b} | | | | | | | | | |
| Update list of prior | | X | X | X | X | X | X | X | X |
| and concomitant | | | | | | | | | |
| medications | | | | | | | | | |
| Ask about AE and | | X | X | X | X | X | X | X | X |
| SAE | | | | | | | | | |
| Assessment of | | X | X | X | X | X | X | X | X |
| COV1D-19 symptoms | | | | | | | | | |
| Swabs for RT-PCR | | | | | X | | X | | X |
| Vitals in-clinic^{c} | | | | | | | | | X |
| Physical Exam | | | | | | | | | X |

Regarding Table 10: The Zinc may be reduced to 25 mg if GI upset occurs, phone/video calls to the patient occur weekly during Weeks 2-11, and vitals in-clinic include height, weight, blood pressure (following 5 minutes sitting) pulse, respiratory rate, temperature, and oxygen saturation.

Statistical Analysis: The treated patients in this study are compared to the placebo group. Measurements include PCR test results, presence or absence of symptoms, and symptom severity.

The change of these measurements from the end to the baseline (post-pre) are used as the primary outcome, for example, μe=μe1−μe0, where μe1 and μe0 are the outcome of patients from the treatment group at the end and at baseline, respectively.

H₀:Δ≤δ₀ against H_{α}:δ>δ₀ where δ0 is clinically meaningful threshold to measure the disease symptoms. In this study, that meaningful threshold is calculated as mean change in clinical symptoms as recorded in the diary, from baseline through week 12. Each category in the diary is assigned a number, 0 for None, 1 for Mild, 2 for Moderate and 3 for severe. Each category is analyzed independently and as a group.

Categorical variables are summarized by presenting the number (n) and percent (%) of subjects in each category. All Statistical tests for the analysis are performed using the p<0.05 level of significance. All confidence intervals are one-sided.

Since these are healthcare workers who are exposed to COVID-19 at every shift, efficacy is determined by RT-PCR testing, as well as the presence or absence of symptoms as recorded in the patient diary via EDC.

### Example 3: Use of Hydroxychloroquine, Azithromycin, Vitamin C, Vitamin D, and Zinc to Treat COVID-19 Infection

Objectives: test the efficacy of hydroxychloroquine, azithromycin, vitamin C, vitamin D, and zinc in the treatment of patients with COVID-19 infection and to assess the safety and tolerability of this treatment in patients with COVID-19 infection.

Procedure: First, the patient is determined to have COVID-19.

Day 1 following positive test (isolation): The patient takes prescribed regimen outlined in Table 11.

TABLE 11

| | | |
|---|---|---|
| Drug | AM Dose | PM Dose |

| | | | |
|---|---|---|---|
| Hydroxychloroquine | 200 | mg | 200 mg |
| Azithromycin | 500 | mg | — |
| Vitamin C | 3000 | mg | — |
| Vitamin D | 3000 | mg | — |
| Zinc | 50 | mg | — |

Day 2-Day 5: The patient takes the prescribed regimen outlined in Table 12.

TABLE 12

| | | |
|---|---|---|
| Drug | AM Dose | PM Dose |

| | | | |
|---|---|---|---|
| Hydroxychloroquine | 200 | mg | 200 mg |
| Azithromycin | 250 | mg | — |
| Vitamin C | 3000 | mg | — |
| Vitamin D | 3000 | mg | — |
| Zinc | 50 | mg | — |

Day 6-Day 10: The patient takes the prescribed regimen outlined in Table 13.

TABLE 13

| Drug | AM Dose | PM Dose |
|---|---|---|
| Hydroxychloroquine | 200 mg | 200 mg |
| Vitamin C | 3000 mg | — |
| Vitamin D | 3000 mg | — |
| Zinc | 50 mg | — |

Follow-Up Period: Month 1 (outpatient only AFTER negative test): vital signs are taken that include blood pressure, heart rate, respiratory rate, oxygen saturation, temperature), the patient is assessed for adverse events and serious adverse events, and swabs for PCR are taken.

Month 3 (outpatient): vital signs are taken that include blood pressure, heart rate, respiratory rate, oxygen saturation, temperature), the patient is assessed for adverse events and serious adverse events, and swabs for PCR are taken.

Month 6 (outpatient) vital signs are taken that include blood pressure, heart rate, respiratory rate, oxygen saturation, temperature), the patient is assessed for adverse events and serious adverse events, and swabs for PCR are taken.

Month 9 (outpatient) vital signs are taken that include blood pressure, heart rate, respiratory rate, oxygen saturation, temperature), the patient is assessed for adverse events and serious adverse events, and swabs for PCR are taken.

Month 12 (outpatient) vital signs are taken that include blood pressure, heart rate, respiratory rate, oxygen saturation, temperature), the patient is assessed for adverse events and serious adverse events, and swabs for PCR are taken.

COVID-19 sample collection procedure is as follows: nasopharyngeal (NP) and oropharyngeal (OP) swabs are collected according to CDC protocol. The swabs comprise synthetic fiber swabs with plastic shafts. NP swabs are collected by insertion of a swab into the nostril parallel to the palate. The swab is left in place a few seconds to allow it to absorb secretions. OP swabs are inserted into the oropharynx parallel to the palate, avoiding the tongue. The swab is left in place a few seconds to allow it to absorb secretions. NP and OP swabs are immediately placed in sterile tubes with 2-3 mL of viral transport media. The tubes are placed in biohazard bags then boxes and couriered to the local Public Health Lab. Table 14 outlines the schedule of events for Example 3.

TABLE 14

| | Diagnosis and Rx | | Month 1 | Month 3 | Month 6 | Month 9 | Month 12 |
|---|---|---|---|---|---|---|---|
| Assessment | (Day 1) | Day 1→ 7 | (±4 d) | (±4 d) | (±4 d) | (±4 d) | (±4 d) |
| Informed Consent & Demographics | X | | | | | | |
| Confirmation of Positive PCR | X | | | | | | |
| for COVID-19 | | | | | | | |
| Review of Medical Records | X | | | | | | |
| Vitals^{a} | | | X | X | X | X | X |
| Prescription of Antimicrobials^{b} | X | | | | | | |
| Ask about AE and SAE | | X | X | X | X | X | X |

Regarding Table 14: Vitals to include height (only at first visit), weight, blood pressure (following 5 minutes sitting) pulse, respiratory rate, temperature, and oxygen saturation. Dosage to be given as in section 8, below

Table 15 provides a summary of the antimicrobial dosage of Example 3.

TABLE 15

| | | | | Fre- |
|---|---|---|---|---|
| Treatment Method | Medication | Dose | | quency |
| Quintuple Therapy | Hydroxychloroquine | 200 | mg | BID |
| | Azithromycin | 500 mg\Day | | Daily |
| | | 1→250 mg Day 2-5 | | |
| | Vitamin C | 3000 | mg | Daily |
| | Vitamin D | 5000 | mg | Daily |
| | Zinc | 50 | mg | Daily |

### Example 4: Randomized, Double-Blind, Placebo-Controlled Phase IIA Study of Hydroxychloroquine, Azithromycin, Vitamin C, Vitamin D, and Zinc to Treat COVID-19 Infection

Objectives: Test the efficacy of hydroxychloroquine, azithromycin, vitamin C, vitamin D, and zinc in the treatment of patients with COVID-19 infection and to assess the safety and tolerability of this treatment in patients with COVID-19 infection.

Procedure: First, the patient's diagnosis of COVID-19 infection is confirmed.

Screening Period (Days −3 to −1): Prescription of home health monitoring equipment that includes a thermometer, a pulse oximeter, a pregnancy test if applicable, and a daily diary. There are two groups being studied: Arm 1 and Arm 2.

Arm 1 is prescribed the following antimicrobials: Hydroxychloroquine 200 MG BID for 10 days; Azithromycin 500 mg on day 1, 250 mg day 2-5; Vitamin C 3000 mg for 10 days, then 1500 mg for 20 days; Vitamin D 3000 IU for 10 days, then 1500 IU for 20 days; and Zinc 50 mg for 10 days, then 25 mg for 20 days.

Arm 2 is prescribed the following antimicrobials: Placebo for Hydroxychloroquine BID for 10 days; Placebo for Azithromycin to be taken 2 the on Day 1, then 1 on Days 2-5; Vitamin C 3000 mg for 10 days, then 1500 mg for 20 days; Vitamin D 3000 IU for 10 days, then 1500 IU for 20 days; and Zinc 50 mg for 10 days, then 25 mg for 20 days. Table 16 outlines the prescribed antimicrobials discussed above.

TABLE 16

| | | | | Fre- |
|---|---|---|---|---|
| Treatment Method | Medication | Dose | | quency |
| Quintuple Therapy | Hydroxychloroquine | 200 | mg | BID |
| | Azithromycin | 500 mg\Day | | Daily |
| | | 1→250 mg Day 2-5 | | |
| | Vitamin C | 3000 | mg | Daily |
| | Vitamin D | 5000 | mg | Daily |
| | Zinc | 50 | mg | Daily |
| Placebo | Placebo | 1 tablet | | BID |
| | Placebo | 2 tablets day 1, then | | Daily |
| | | 1 tablet day 2-5 | | |
| | Vitamin C | 3000 | mg | Daily |
| | Vitamin D | 3000 | IU | Daily |
| | Zinc | 50 | mg | Daily |

Treatment Period: Day 1 following positive test (isolation), the patient is video called to ensure they have all study materials and the following is discussed: Use of home health equipment, Subject will take baseline measurements at this time and record it in the diary, Diary and how to transmit its contents, Medication dosing, Subject will take pregnancy test if applicable, Subject will use provided equipment to measure vital signs such as EKG, Oxygen Saturation, and Temperature. The patient takes prescribed regimen outlined in Table 17.

TABLE 17

| | | |
|---|---|---|
| Drag | AM Dose | PM Dose |

| | | | |
|---|---|---|---|
| Hydroxychloroquine (or Placebo) | 200 | mg | 200 mg |
| Azithromycin (or Placebo) | 500 | mg | — |
| Vitamin C | 3000 | mg | — |
| Vitamin D | 3000 | IU | — |
| Zinc | 50 | mg | — |

Day 2: The patient completes the AM and PM diary entries, the patient uses the provided equipment to measure vital signs such as EKG, Oxygen saturation, and temperature, and the patient takes the prescribed regimen outlined in Table 18.

TABLE 18

| | | |
|---|---|---|
| Drug | AM Dose | PM Dose |

| | | | |
|---|---|---|---|
| Hydroxychloroquine (or Placebo) | 200 | mg | 200 mg |
| Azithromycin (or Placebo) | 250 | mg | — |
| Vitamin C | 3000 | mg | — |
| Vitamin D | 3000 | IU | — |
| Zinc | 50 | mg | — |

Day 3: The patient is called and asked if they are experiencing any difficulties with swab collection, whether there have been any adverse events and/or serious adverse events, the list of prior and concomitant medications is updated, the patient is asked about symptom resolution or progression, the patient is instructed on how to collect nasal swabs, and the patient then collects the first nasal swab. The patient completes their AM and PM diary entries, and collects their vital signs such as EKG, oxygen saturation and temperature. The patient takes the prescribed regimen outlined in Table 19.

TABLE 19

| | | |
|---|---|---|
| Drug | AM Dose | PM Dose |

| | | | |
|---|---|---|---|
| Hydroxychloroquine (or Placebo) | 200 | mg | 200 mg |
| Azithromycin (or Placebo) | 250 | mg | — |
| Vitamin C | 3000 | mg | — |
| Vitamin D | 3000 | IU | — |
| Zinc | 50 | mg | — |

Day 4: The patient completes their AM and PM diary entries, collects their vital signs, and takes the prescribed regimen outlined in Table 20.

TABLE 20

| | | |
|---|---|---|
| Drug | AM Dose | PM Dose |

| | | | |
|---|---|---|---|
| Hydroxychloroquine (or Placebo) | 200 | mg | 200 mg |
| Azithromycin (or Placebo) | 250 | mg | — |
| Vitamin C | 3000 | mg | — |
| Vitamin D | 3000 | IU | — |
| Zinc | 50 | mg | — |

Day 5: The patient completes their AM and PM diary entries, collects their vital signs, collects a nasal swab, and takes the prescribed regimen outlined in Table 21.

TABLE 21

| | | |
|---|---|---|
| Drug | AM Dose | PM Dose |

| | | | |
|---|---|---|---|
| Hydroxychloroquine (or Placebo) | 200 | mg | 200 mg |
| Azithromycin (or Placebo) | 250 | mg | — |
| Vitamin C | 3000 | mg | — |
| Vitamin D | 3000 | IU | — |
| Zinc | 50 | mg | — |

Day 6: The patient completes their AM and PM diary entries, collects their vital signs, and takes the prescribed regimen outlined in Table 22.

TABLE 22

| | | |
|---|---|---|
| Drug | AM Dose | PM Dose |

| | | | |
|---|---|---|---|
| Hydroxychloroquine (or Placebo) | 200 | mg | 200 mg |
| Vitamin C | 3000 | mg | — |
| Vitamin D | 3000 | IU | — |
| Zinc | 50 | mg | — |

Day 7: The patient is called and asked whether there have been any adverse events and/or serious adverse events, the list of prior and concomitant medications is updated, the patient is asked about symptom resolution or progression, and the patient then collects a nasal swab. The patient also completes their AM and PM diary entries and collects their vital signs. The patient takes the prescribed regimen outlined in Table 23.

TABLE 23

| | | |
|---|---|---|
| Drug | AM Dose | PM Dose |

| | | | |
|---|---|---|---|
| Hydroxychloroquine (or Placebo) | 200 | mg | 200 mg |
| Vitamin C | 3000 | mg | — |
| Vitamin D | 3000 | IU | — |
| Zinc | 50 | mg | — |

Day 8: The patient completes their AM and PM diary entries, collects their vital signs, and takes the prescribed regimen outlined in Table 24.

TABLE 24

| | | |
|---|---|---|
| Drug | AM Dose | PM Dose |

| | | | |
|---|---|---|---|
| Hydroxychloroquine (or Placebo) | 200 | mg | 200 mg |
| Vitamin C | 3000 | mg | — |
| Vitamin D | 3000 | IU | — |
| Zinc | 50 | mg | — |

Day 9: The patient completes their AM and PM diary entries, collects their vital signs, and takes the prescribed regimen outlined in Table 25.

TABLE 25

| | | |
|---|---|---|
| Drug | AM Dose | PM Dose |

| | | | |
|---|---|---|---|
| Hydroxychloroquine (or Placebo) | 200 | mg | 200 mg |
| Vitamin C | 3000 | mg | — |
| Vitamin D | 3000 | IU | — |
| Zinc | 50 | mg | — |

Day 10: The patient is called reminded to decrease dosage of vitamins C, vitamin D, and zinc tomorrow. The patient is asked whether there have been any adverse events and/or serious adverse events, the list of prior and concomitant medications is updated, and the patient is asked about symptom resolution or progression. The patient also completes their AM and PM diary entries and collects their vital signs. The patient takes the prescribed regimen outlined in Table 26.

TABLE 26

| | | |
|---|---|---|
| Drug | AM Dose | PM Dose |

| | | | |
|---|---|---|---|
| Hydroxychloroquine (or Placebo) | 200 | mg | 200 mg |
| Vitamin C | 3000 | mg | — |
| Vitamin D | 3000 | IU | — |
| Zinc | 50 | mg | — |

Day 11 to Day 13: The patient completes their AM and PM diary entries and takes the prescribed regimen outlined in Table 27.

TABLE 27

| | | |
|---|---|---|
| Drug | AM Dose | PM Dose |

| | | | |
|---|---|---|---|
| Vitamin C | 1500 | mg | — |
| Vitamin D | 1500 | IU | — |
| Zinc | 25 | mg | — |

Day 14: The patient is called and asked whether there have been any adverse events and/or serious adverse events, the patient is asked about symptom resolution or progression, and the patient then collects a nasal swab. The patient also completes their AM and PM diary entries and collects their vital signs. The patient takes the prescribed regimen outlined in Table 28.

TABLE 28

| | | |
|---|---|---|
| Drug | AM Dose | PM Dose |

| | | | |
|---|---|---|---|
| Vitamin C | 1500 | mg | — |
| Vitamin D | 1500 | IU | — |
| Zinc | 25 | mg | — |

Day 15 to Day 30: The patient completes their AM and PM diary entries and takes the prescribed regimen outlined in Table 29.

TABLE 29

| | | |
|---|---|---|
| Drug | AM Dose | PM Dose |

| | | | |
|---|---|---|---|
| Vitamin C | 1500 | mg | — |
| Vitamin D | 1500 | IU | — |
| Zinc | 25 | mg | — |

Follow-Up Period: Month 1 (outpatient only AFTER negative test): Vital signs are taken (BP, HR, RR, oxygen saturation, temperature), the patient is assessed for adverse events and/or serious adverse events, and EKG is administered as well as physical exam and a blood draw for CBC/Complete metabolic panel/C-Reactive Protein. The list of prior and concomitant medications is updated and a nasal swab is collected.

Month 2 (outpatient): Vital signs are collected (BP, HR, RR, oxygen saturation, temperature) the patient is assessed for adverse events and/or serious adverse events, and EKG is administered as well as physical exam and a blood draw for CBC/Complete metabolic panel/C-Reactive Protein. The list of prior and concomitant medications is updated.

Month 3 (outpatient): Vital signs are taken (BP, HR, RR, oxygen saturation, temperature), the patient is assessed for adverse events and/or serious adverse events, and EKG is administered as well as physical exam and a blood draw for CBC/Complete metabolic panel/C-Reactive Protein. The list of prior and concomitant medications is updated and a nasal swab is collected.

COVID-19 Sample Collection Procedure is the same as that outlined above in prior Examples. Table 30 outlines the schedule of events for Example 4.

TABLE 30

| | Screening | | | | | | | | | | |
|---|---|---|---|---|---|---|---|---|---|---|---|
| | (Da −3 to | | | | | | | | | | |
| Assessment | Day −1) | Day 1 | Day 2 | Day 3 | Day 4 | Day 5 | Day 6 | Day 7 | Day 8 | Day 9 | Day 10 |
| Informed Consent & | X | | | | | | | | | | |
| Demographics | | | | | | | | | | | |
| Make list of prior | X | | | x | | | | x | | | x |
| and concomitant | | | | | | | | | | | |
| medications | | | | | | | | | | | |
| Confirmation of | X | | | | | | | | | | |
| Positive PCR for | | | | | | | | | | | |
| COVID-19 | | | | | | | | | | | |
| Review of Medical | X | | | | | | | | | | |
| Records | | | | | | | | | | | |
| Prescription of | X | | | | | | | | | | |
| Antimicrobials^{a} | | | | | | | | | | | |
| Provide home health | X | | | | | | | | | | |
| equipment^{b} | | | | | | | | | | | |
| Pregnancy test^{c} | | X | | | | | | | | | |
| Call Subject at home^{d} | | X | | X | | | | X | | | X |
| Vitals at home^{e} | | X | X | X | X | X | X | X | X | X | X |
| Subject will complete | | X | x | x | x | x | x | x | x | x | x |
| AM/PM diary | | | | | | | | | | | |
| Hydroxychloroquine | | X | X | X | X | X | X | X | X | X | X |
| 200 mg BID (or | | | | | | | | | | | |
| placebo | | | | | | | | | | | |
| Azithromycin 500 mg | | X | | | | | | | | | |
| (2 tablets) once | | | | | | | | | | | |
| daily (or placebo) | | | | | | | | | | | |
| Azithromycin 250 mg | | | X | X | X | X | | | | | |
| (1 tablet) once | | | | | | | | | | | |
| daily (or placebo) | | | | | | | | | | | |
| Vitamin C 3000 mg | | | X | X | X | X | X | X | X | X | X |
| (4 capsules) once | | | | | | | | | | | |
| daily | | | | | | | | | | | |
| Vitamin D 3000 IU | | | X | X | X | X | X | X | X | X | X |
| (4 capsules) once | | | | | | | | | | | |
| daily | | | | | | | | | | | |
| Zinc 50 mg | | | X | X | X | X | X | X | X | X | X |
| (4 capsules) once | | | | | | | | | | | |
| daily | | | | | | | | | | | |
| Vitamin C 1500 mg | | | | | | | | | | | |
| (2 capsules) once | | | | | | | | | | | |
| daily | | | | | | | | | | | |
| Vitamin D 1500 IU | | | | | | | | | | | |
| (2 capsules) once | | | | | | | | | | | |
| daily | | | | | | | | | | | |
| Zinc 25 mg | | | | | | | | | | | |
| (2 capsules) once | | | | | | | | | | | |
| daily | | | | | | | | | | | |
| Ask about AE and | | | | X | | | | X | | | X |
| SAE | | | | | | | | | | | |
| Vitals in-clinic^{f} | | | | | | | | | | | |
| Physical exam | | | | | | | | | | | |
| EKG in-clinic | | | | | | | | | | | |
| Swabs for RT-PCR | | | | X | | X | | X | | | |
| Bloodwork^{g} | | | | | | | | | | | |
| Review list of | | X | X | X | X | X | X | X | X | X | X |
| prior and | | | | | | | | | | | |
| concomitant | | | | | | | | | | | |
| medications | | | | | | | | | | | |

| Assessment | Day 11 | Day 12 | Day 13 | Day 14 | Days 15-30 | Month 1 | Month 2 | Month 3 |
|---|---|---|---|---|---|---|---|---|
| Informed Consent & | | | | | | | | |
| Demographics | | | | | | | | |
| Make list of prior | | | | x | | X | X | X |
| and concomitant | | | | | | | | |
| medications | | | | | | | | |
| Confirmation of | | | | | | | | |
| Positive PCR for | | | | | | | | |
| COVID-19 | | | | | | | | |
| Review of Medical | | | | | | | | |
| Records | | | | | | | | |
| Prescription of | | | | | | | | |
| Antimicrobials^{a} | | | | | | | | |
| Provide home health | | | | | | | | |
| equipment^{b} | | | | | | | | |
| Pregnancy test^{c} | | | | | | | | |
| Call Subject at home^{d} | | | | X | | | | |
| Vitals at home^{e} | | | | | | | | |
| Subject will complete | x | x | x | x | x | | | |
| AM/PM diary | | | | | | | | |
| Hydroxychloroquine | | | | | | | | |
| 200 mg BID (or | | | | | | | | |
| placebo | | | | | | | | |
| Azithromycin 500 mg | | | | | | | | |
| (2 tablets) once | | | | | | | | |
| daily (or placebo) | | | | | | | | |
| Azithromycin 250 mg | | | | | | | | |
| (1 tablet) once | | | | | | | | |
| daily (or placebo) | | | | | | | | |
| Vitamin C 3000 mg | | | | | | | | |
| (4 capsules) once | | | | | | | | |
| daily | | | | | | | | |
| Vitamin D 3000 IU | | | | | | | | |
| (4 capsules) once | | | | | | | | |
| daily | | | | | | | | |
| Zinc 50 mg | | | | | | | | |
| (4 capsules) once | | | | | | | | |
| daily | | | | | | | | |
| Vitamin C 1500 mg | X | X | X | X | X | | | |
| (2 capsules) once | | | | | | | | |
| daily | | | | | | | | |
| Vitamin D 1500 IU | X | X | X | X | X | | | |
| (2 capsules) once | | | | | | | | |
| daily | | | | | | | | |
| Zinc 25 mg | X | X | X | X | X | | | |
| (2 capsules) once | | | | | | | | |
| daily | | | | | | | | |
| Ask about AE and | | | | X | | X | X | X |
| SAE | | | | | | | | |
| Vitals in-clinic^{f} | | | | | | X | X | X |
| Physical exam | | | | | | X | X | X |
| EKG in-clinic | | | | | | X | X | X |
| Swabs for RT-PCR | | | | X | | X | | X |
| Bloodwork^{g} | | | | | | X | X | X |
| Review list of | X | X | X | x | X | X | X | x |
| prior and | | | | | | | | |
| concomitant | | | | | | | | |
| medications | | | | | | | | |

Regarding Table 30: The antimicrobials include hydroxychloroquine 200 mg tablets (#20), azithromycin 250 mg tablets (#6), vitamin C 750 mg capsules (#80), vitamin D 750 IU capsules (#80), zinc 12.5 mg capsules (#80); the home health equipment includes an EKG (worn continuously), pulse oximeter, and thermometer; the pregnancy test is administered if the patient is a woman of childbearing potential; the patients are called at home to remind them to collect swabs for RT-PCT, ask about AE/SAE, ask about symptoms, and answer any questions; the vitals taken at home include an EKG, oxygen saturation, and temperature; the vitals in-clinic include height (only at first visit), weight, blood pressure (following 5 minutes sitting) pulse, respiratory rate, temperature, and oxygen saturation; and the bloodwork includes CBC, Complete Metabolic Panel, and CRP (details in section 9 Sample Collection).

Statistical Analysis: The treated patients in this study are compared to the placebo group. Measurements include PCR test results, presence or absence of symptoms, and symptom severity. PCR results will be compared between the groups as positive or negative

In this study, the meaningful threshold is calculated as mean change in clinical symptoms as recorded in the diary, from Day 1 through week 12. Each category in the diary is assigned a number, 0 for None, 1 for Mild, 2 for Moderate and 3 for severe. Each category is analyzed independently and as a group. Ultimately, efficacy is determined based upon reduction and/or progression of symptomatic days, reduction of symptom severity, as well as analysis of the subject's RT-PCR testing per protocol. These data are compared to an existing database of de-identified Subject data.

The change of these measurements from the end to the baseline (post-pre) are used as the primary outcome, for example, μe=μₑ₁−μₑ₀, where μₑ₁ and μₑ₀ are the outcome of Subjects from the treatment group at the end and at baseline, respectively.

Categorical variables are summarized by presenting the number (n) and percent (%) of subjects in each category. All Statistical tests for the analysis are performed using the p<0.05 level of significance. All confidence intervals will be one-sided

Sample size was calculated as follows:

$$n = \frac{log B}{log ρ}$$

Where β9 =the probability of a Type II error

p=the proportion of the population NOT affected

The proportion of the population affected by COVID-19 is 0.005 percent, thus 0.995 percent aren't affected

The probability of a type II error is 0.05

Thus:

$$n = \frac{log 0.050}{log 0.9 \; 95}$$

$$n = 597.647$$

A sample size of 600 was used.

### Example 5: Successful Treatment of COVID-19 Infected Outpatients and Prophylaxis of Immediate Associates

Objective: to successfully treat COVID-19 infected outpatients and prophylaxis of immediate associates.

Procedure: Prospective COVID-19 infected individuals were diagnosed using a Pangea DNA/RNA Shield™ Collection Tube to obtain a nasopharyngeal swab and PCR +ve patients were entered into the study. They were immediately commenced on a 10 day course of Hydroxychloroquine (200 mg, twice a day, for 10 days), Azithromycin extended release (500 mg on day 1, then 250 mg a day for days 9-10), zinc (50 mg a day for days 1-10), Vitamin D (3000 IU a day for days 1-10) and Vitamin C (3000 mg a day for days 1-10). Some individuals lived alone, otherwise immediate partners and family deemed to be most exposed were given a prophylactic which comprised hydroxychloroquine 200 mg twice a day on day 1 only with Zinc, Vitamin C and Vitamin D for given at the same doses as above for days 1-10.

Results: In 11 families a total of 21 family members were identified to be PCR COVID-19 positive index cases and were treated with the above treatment protocol while 22 exposed associates with negative PCR were given the above prophylaxis protocol. This is shown below in Table 31. All 21 index cases were cured of COVID-19 infection as judged by the repeat swab PCR on day 10 and accompanying symptom resolution. None of the 22 highly exposed associates developed COVID-19 infection when retested on day 10 (day 14 in Family 10) in spite of close co-habitation with the infected index cases. TABLE 31: Results from families received 10-day course of daily HCQ (200 mg bd), AZ extended release (500 mg day 1, then 250 mg), zinc (50 mg), Vitamin D (3000 IU) and Vitamin C (3000 mg)

TABLE 31

| | | | | After treating | |
|---|---|---|---|---|---|
| | | | | with HAZDPAC | After treating |
| | | Age (years)- | | Cured/PCR test | ZINCD + H |
| | Patient/s | sex of the | Comorbidities of the | positive/negative/ | family member |
| Family | treated | patient | patients | other symptoms | prophylaxed |
| #1 | 1 | 23 - male | Asthma | Negative PCR - | Parents |
| | | | | day 10 | overweight, |
| | | | | | diabetes, father |
| | | | | | with heart |
| | | | | | disease never got |
| | | | | | the disease |
| #2 | 3 | 60- male | Asthma | All 3 patients | Mother didn't |
| | | (father) | | cured | get the virus |
| | | 18- male | | | |
| | | (son) | | | |
| | | 16- female | | | |
| | | (daughter) | | | |
| #3 | 1 | 40-feniale | Avoided intubation | Sever multiple | Husband did not |
| | | | by leaving hospital | symptoms | turn positive |
| | | | | resolved at home | |
| #4 | 2 | 78- male | BCG + COPD, | Both cured | Daughter, son- |
| | | | diabetes, heart | | in-law and 2 |
| | | | disease (heart | | grandkids didn't |
| | | | surgery month prior | | catch the virus |
| | | 77- female | Pacemaker, BCG | | |
| #5 | 3 | 56-male | Unable to eat or | Started treatment | Wife never got |
| | | | drink | on day 10, Sever | the disease |
| | | | | symptoms | |
| | | | | resolved | |
| | | 27- male | Asthma | Cured | |
| | | 24- female | | | |
| #6 | 1 | 56- female | | Admitted to | Husband never |
| | | | | hospital -sent | got the disease |
| | | | | home, as two | |
| | | | | kids were sick | |
| | | | | with fever. All | |
| | | | | recovered with | |
| | | | | vitamins | |
| #7 | 1 | 44-female | | Cured, | Husband and 4 |
| | | | | autoimmune | kids never got |
| | | | | issues started | the disease |
| #8 | 3 | 52- female | Lupus | Cured | Boyfriend (54 y) |
| | | 53- male | | Cured | with Diabetes, |
| | | 19- male | Severe asthma | Cured | son (18) and |
| | | | | | daughter (16) |
| | | | | | never got the |
| | | | | | disease |
| #9 | 2 | 45-female | | Both cured from | Husband and |
| | | 16-male | | severe symptoms | daughter never |
| | | | | of cough and | got the disease |
| | | | | fever | |
| #10 | 2 | 44- male | | Cured from loss | Mother (50 y) |
| | | | | of smell, fever, | never got the |
| | | | | cough | disease |
| | | 25- female | | Cured | |
| | | 21-female | | Partially treated | |
| | | | | with HAZDPAC - | |
| | | | | slowly turned −ve | |
| #11 | 1 | 33-female | No | ICU nurse | Boyfriend never |
| | | | | recovered | got the disease |

Apart from the families a further 11 single infected individuals found to be swab PCR positive were treated with the above treatment protocol. This is shown below in Table 32. All were also successfully cured of the infection.

TABLE 32

| | | | After |
|---|---|---|---|
| | Age (years)- | | treating with |
| | sex of the | Comorbidities/symptoms | HAZDPAC |
| Patient | patient | of the patients | Cured/not |
| #1 | 44-female | Asthma | Cured |
| | (no BCG in | | |
| | childhood) | | |
| #2 | 81- male | Diarrhea | Cured |
| #3 | 52-female | Fever | Cured |
| #4 | 66-male | Valve surgery | Cured |
| #5 | 29-female | Asthma | Cured |
| | (no BCG in | | |
| | childhood) | | |
| #6 | 50-female | Auto immune thyroiditis, sever | Cured |
| | | cough and fever | |
| #7 | 43-male | Cough, desaturation of oxygen | Cured |
| | 53-male | Diarrhea, cough, fever, | Cured |
| | | desaturation of oxygen | |
| #9 | 44- female | Autoimmune history, fever, | Cured |
| | | increase heart rate | |
| #10 | 44- female | Pneumothorax discharged from | Cured |
| | | ICU with COVID-19 | |
| #11 | 43- male | Fever +ve Covid-19 PCR | Cured |

A further 9 individuals recently closely exposed to Covid-19 infected persons were given the prophylaxis protocol outlined above. The prophylactic worked very well with no exposed person acquiring the infection. This is shown in Table 33.

TABLE 33

| Individual | Age (years) & details of the individual | After treating with ZINCD + H |
|---|---|---|
| #1 | Mother of 16 year old child who had | Prophylaxed and never got the |
| | COVID-19 | disease |
| #2 | 24- female, ICU nurse - multiple | Prophylaxed and never got the |
| | exposures | disease |
| #3 | 47- male, cardiologist exposed to +ve | Prophylaxed and never got the |
| | patients | disease |
| #4 | 70- male medical director of a hospital | Prophylaxed and never got the |
| | (exposed to numerous doctors with | disease |
| | COVID-19) | |
| #5 | 55- male anaesthesiologist (intubates | Prophylaxed and never got the |
| | COVID-19 patients) | disease |
| #6 | 55- ICU nurse (worked on COVID-19 | Prophylaxed and never got the |
| | floor) | disease |
| #7 | 40- ICU nurse many Covid-19 patients | Prophylaxed and never got the |
| | | disease |
| #8 | 53-Doctor with pancreatitis | Prophylaxed and never got the |
| | | disease |
| #9 | 28-Paramedic - healthy | Prophylaxed and never got the |
| | | disease |

Discussion: It was demonstrated that a 10 day combination of hydroxychloroquine, azithromycin (for 5 days only), zinc with vitamin D and vitamin C, can result in uniform cure of COVID-19 infection when used in an outpatient population. The prophylaxis treatment noted above for those closely exposed to proven, infected patients can completely prevent spread of COVID-19. This combination of test-and-treat permits abolishing of new outbreaks of infection such as a ‘next wave’—by avoiding quarantine to treat the infected and give prophylactics to surrounding staff and family.

In conclusion, this is an effective anti-Covid-19 therapy as well as an effective prophylactic combination capable of arresting the spread of coronavirus infection throughout the community. This is achieved by treating the index case and the surrounding associates of the patient as early as possible after infection is identified and then treating the people they live with and close associates.

### Example 6: Presence of the SARS-CoV-2 by NGS of Fecal Samples

Objective: In view of the large percentage of SARS-CoV-2 detectible by RT-PCR in stools of infected patients, the objective was to identify the presence of the SARS-CoV-2 by NGS of fecal samples from symptomatic study participants positive for SARS-CoV-2 by nasopharyngeal sample RT-PCR, in addition to asymptomatic individuals (with or without prior nasopharyngeal sample RT-PCR). The objective was also to execute whole genome analysis to characterize SARS-CoV-2 mutational variations to identify potentially significant nucleotide changes.

Procedure: Study participants (n=14) underwent testing for SARS-CoV-2 from fecal samples by whole genome enrichment NGS. Following fecal collection (Zymo Research Shield Fecal Collection Tubes), RNA was extracted (Qiagen Allprep Power Viral Kit), reverse transcribed (New England Biolabs NEBNext 1st and 2nd Strand Synthesis Modules), library prepped (Illumina Nextera Flex for Enrichment), enriched (Ilumina Respiratory Virus Oligo Panel), and sequenced on Illumina's NextSeq 550 System. Sequences were then mapped to the SARS-CoV-2 Wuhan-Hu-1 (MN90847.3) complete genome utilizing One Codex's SARS-CoV-2 bioinformatics analysis pipeline. SARS-CoV-2 positive samples were further analyzed for mutational variants that differed from the reference genome. Of the 14 study participants, 12 also had their nasopharyngeal swabs tested for SARS-CoV-2 by RT-PCR.

Results: The results from patients that had their stool samples tested by whole genome enrichment NGS, and their nasopharyngeal swabs tested by RT-PCR for the presence of SARS-CoV-2 were evaluated. Of the 14 study participants, ten were symptomatic and tested positive for SARS-CoV-2 by RT-PCR, two asymptomatic individuals tested negative, and two other asymptomatic individuals did not undergo RT-PCR testing (Table 34). Patients 5 and 7, which tested positive by RT-PCR from nasopharyngeal swabs, were treated with the protocol from Example 5 above (Hydroxychloroquine, Azithromycin, vitamin C, vitamin D, and zinc for 10 days prior to fecal collection). Similarly, after positive nasopharyngeal swab, patient 13 was treated with vitamin C, vitamin D, and zinc for 10 days (the same protocol as noted above in Example 5) before fecal collection. The concordance of SARS-CoV-2 detection by enrichment NGS from stools among positive non-treated patients tested by RT-PCR nasopharyngeal analysis was 100% (7/7). Patient 8, who did not undergo nasopharyngeal analysis, tested positive for SARS-CoV-2 by NGS. The three patients (5, 7, 13) that received treatment prior to providing fecal samples, all tested negative by NGS. Asymptomatic patients 2 and 9, who tested negative by nasopharyngeal swab, were also negative by NGS, as was asymptomatic patient 14. Table 34 outlines the symptoms and SARS-CoV-2 testing results.

TABLE 34

| | | Nasopharyngeal | | Fecal | Patient |
|---|---|---|---|---|---|
| Sample ID | Symptoms | Swab (RT-PCR) | Treated | (NGS) | Location |
| Patient 1 | febrile, diarrhea, anosmia, O2 sat. <90% | + | no | + | PA |
| Patient 3 | febrile, diarrhea, O2 sat. <90% | + | no | + | CA |
| Patient 4 | febrile, diarrhea, anosmia, O2 sat. <90% | + | no | + | AZ |
| Patient 6 | febrile, cough, anosmia | + | no | + | AZ |
| Patient 8 | none | n/a | no | + | CA |
| Patient 10 | febrile, cough, headache | + | no | + | GA |
| Patient 11 | febrile, cough, headache | + | no | + | GA |
| Patient 12 | febrile, cough | + | no | + | GA |
| Patient 5 | febrile, cough | + | yes | − | CA |
| Patient 7 | febrile, cough | + | yes | − | GA |
| Patient 13 | febrile, cough | + | yes | − | GA |
| Patient 2 | none | − | no | − | CA |
| Patient 9 | none | − | no | − | CA |
| Patient 14 | none | n/a | no | − | CA |

All fecal samples analyzed by enrichment NGS from positive patients by RT-PCR, achieved 100% genome coverage of SARS-CoV-2 except for patient 3 which had 45%, and patient 10 which had 93% coverage. Table 35 outlines the enrichment NGS metrics.

TABLE 35

| | Genome | # Variants | Mapped | |
|---|---|---|---|---|
| Sample ID | Coverage | (over 10x) | Reads | Mean Depth |
| Patient 1 | 100% | 11 | 465645 | 1129.8x |
| Patient 3 | 45% | 11 | 5984 | 31.7x |
| Patient 4 | 100% | 9 | 131582 | 318.6x |
| Patient 6 | 100% | 10 | 793603 | 1924.6x |
| Patient 8 | 100% | 10 | 496852 | 1206.7 |
| Patient 10 | 93% | 9 | 5929 | 15.6x |
| Patient 11 | 100% | 10 | 1270734 | 3075.3x |
| Patient 12 | 100% | 10 | 38256 | 92.7x |

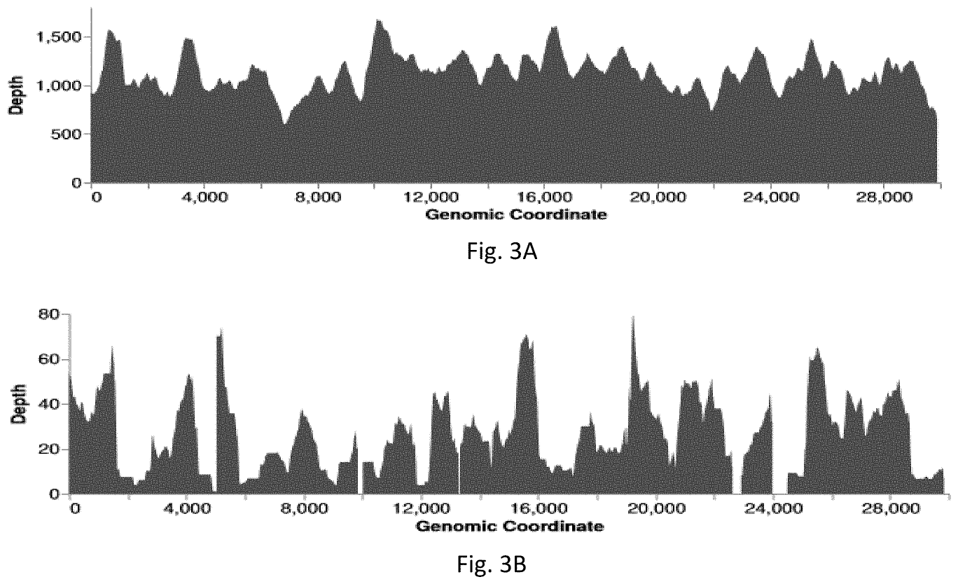

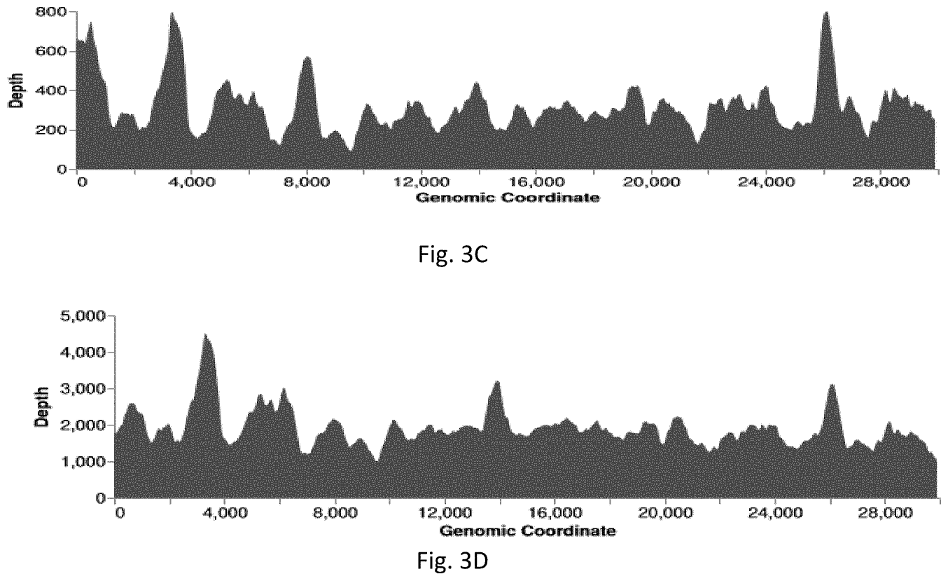

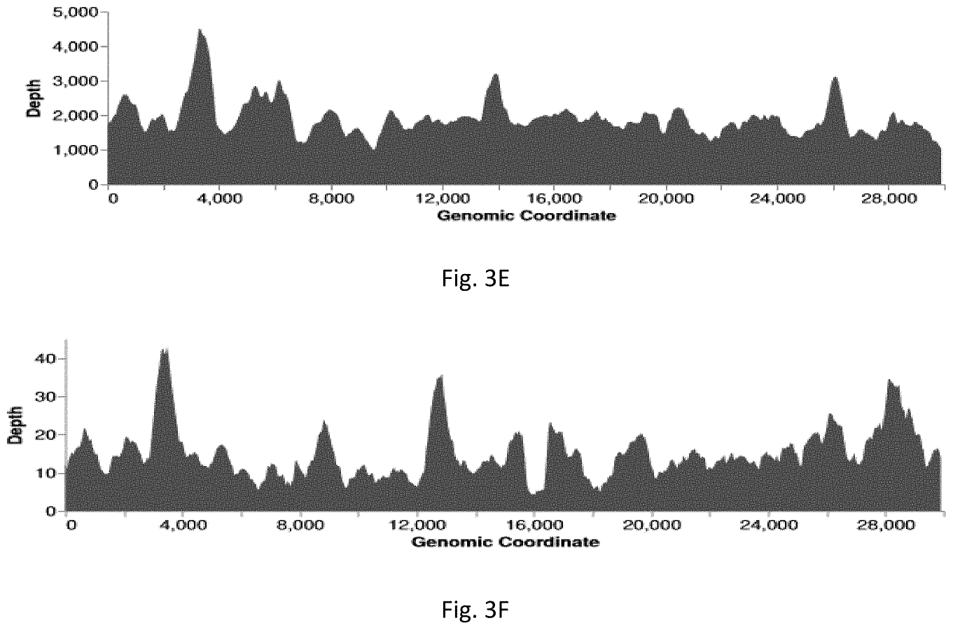

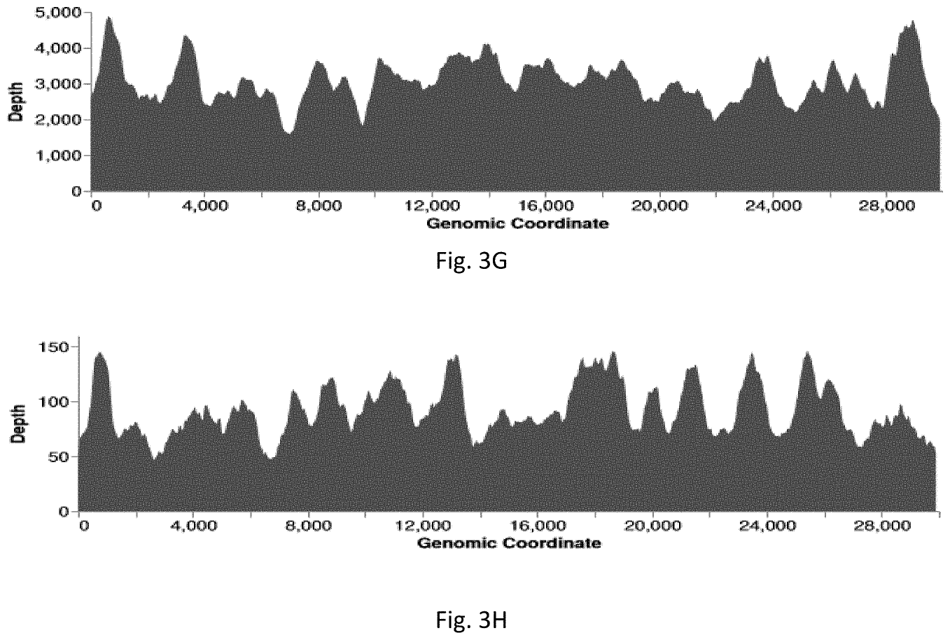

The total number of SARS-CoV-2 mapped reads for patients 1, 3, 4, 6, 8, 10, 11, and 12 were 465645, 5984, 131582, 793603, 496852, 5929, 1270734, and 38256 respectively. The mean read depths of SARS-CoV-2 for patients 1, 3, 4, 6, 8, 10, 11, and 12 were 1129.8x, 31.7x, 318.6x, 1924.6x, 1206.7x, 15.5x, 3075.3x, and 92.7x, and respectively. The read depths at specific coordinates along the SARS-CoV-2 genome for each patient are captured in FIGS. 3A-3H. Whole genome alignment of SARS-CoV-2 in patients 1, 3, 4, 6, 8, 10, 11, and 12 (respectively) as identified by One Codex's SARS-CoV-2 analysis pipeline. The x-axis depicts the genomic coordinates as aligned to the MN908947.3 reference genome, and the y-axis represents the read depth at specific loci.

Following alignment and mapping of SARS-CoV-2, patient genomes were compared to the Wuhan-Hu-1 (MN90847.3) SARS-CoV-2 reference genome via One Codex's bioinformatics pipeline to identify mutational variations. This analysis identified nucleotide variants at positions nt241 (C→T) and nt23403 (A→G) across all positive patients, and variants at positions nt3037 (C→T) and nt25563 (G→T) in seven of the eight patients (Table 3). Interestingly, patients 8, 11, and 12 harbored the same set of variants, as did patients 4 and 6 (who were kindred). Unique variants not identified in any of the other individuals were detected in patients 1, 3, 6, and 10, with patient 3 harboring the most distinct SARS-CoV-2 genome with eight unique variants, followed by patient 1 with seven. Collectively, there were thirty-three different mutations among the patients in which SARS-CoV-2 was detected by whole genome enrichment NGS. Table 36 outlines the SARS-CoV-2 genomic positions, variant changes, and frequencies across the positive patient cohort.

TABLE 36

| Region | | | | | | | | | | |
|---|---|---|---|---|---|---|---|---|---|---|
| (ORF) | Po | Varian | Patien | Patien | Patien | Patien | Patien | Patient | Patient | Patient |
| 5′-UTR | 24 | C → T | 100% | 100% | 100% | 100% | 100% | 100% | 100% | 100% |
| 1a | 83 | T → C | x | x | x | x | 100% | x | 100% | 100% |
| 1a | 10 | C → T | x | x | 100% | 100% | 99% | 100% | 100% | 100% |
| 1a | 17 | C → T | x | x | 100% | 100% | x | x | x | x |
| 1a | 19 | C → T | x | x | x | 87% | x | x | x | x |
| 1a | 30 | C → T | 100% | x | 100% | 100% | 100% | 100% | 100% | 100% |
| 1a | 30 | C → T | x | 89% | x | x | x | x | x | x |
| 1a | 48 | G → T | 75% | x | x | x | x | x | x | x |
| 1a | 67 | C → T | 93% | x | x | x | x | x | x | x |
| 1a | 81 | G → T | x | 100% | x | x | x | x | x | x |
| 1a | 94 | T → C | x | x | x | x | x | 64% | x | x |
| 1a | 94 | T → A | x | x | x | x | x | 64% | x | x |
| 1a | 10 | G → T | x | x | 100% | 100% | x | x | x | x |
| 1a | 11 | G → A | x | x | 100% | 100% | x | x | x | x |
| 1b | 14 | C → T | 100% | x | 100% | 100% | 100% | x | 100% | 100% |
| 1b | 14 | C → T | x | 100% | x | x | x | x | x | x |
| 1b | 16 | C → T | x | x | x | x | 100% | x | 100% | 100% |
| 1b | 16 | C → T | 100% | x | x | x | x | x | x | x |
| 1b | 18 | C → A | x | x | x | x | x | 83% | x | x |
| 1b | 19 | T → G | x | 100% | x | x | x | x | x | x |
| Spike | 21 | T → G | x | 83% | x | x | x | x | x | x |
| Spike | 23 | G → A | x | 75% | x | x | x | x | x | x |
| Spike | 23 | A → G | 100% | 100% | 100% | 100% | 100% | 100% | 100% | 100% |
| Spike | 23 | C → T | 82% | x | x | x | x | x | x | x |
| 3a | 25 | G → T | x | 100% | 100% | 100% | 100% | 100% | 100% | 100% |
| 3a | 25 | C → A | x | x | x | x | 100% | x | 100% | 100% |
| 8 | 27 | C → T | x | x | x | x | 100% | x | 100% | 100% |
| Nucleopr | 28 | G → A | 100% | x | x | x | x | x | x | x |
| Nucleopr | 28 | G → A | 100% | x | x | x | x | x | x | x |
| Nucleopr | 28 | G → C | 100% | x | x | x | x | x | x | x |
| Nucleopr | 28 | C → T | x | 100% | x | x | x | x | x | x |
| Nucleopr | 29 | A → T | x | 100% | x | x | x | x | x | x |
| Nucleopr | 29 | C → G | x | x | x | x | x | 85% | x | x |

Discussion: Although previous studies have identified SARS-CoV-2 in fecal collections by RT-PCR, this study was able to report whole genome sequencing (WGS) of SARS-CoV-2 from stool samples. SARS-CoV-2 was identified in patients that tested positive by nasopharyngeal swab RT-PCR analysis and unique genomes in 62.5% of the NGS positive patients was observed. The overall homology among the genomes was high (99.97%), with variations identified in the ORF regions 1a, 1b, S, 3a, 8, and N. Of particular interest, was the adenine to guanine change in the S protein at position nt23403 which converts aspartic acid to glycine (D→G).

Conclusion: Next generation sequencing identified the SARS-CoV-2 whole genome sequence in 100% of patients with positive nasopharyngeal RT-PCR and did not detect it in treated patients, or those with negative rt-PCR. These results highlight the importance of metagenomic analysis of the SARS-CoV-2 viral genome.

### Example 7: Randomized, Double-Blind, Placebo-Controlled Phase IIA Study of Hydroxychloroquine, Vitamin C, Vitamin D, and Zinc for the Prevention of COVID-19 Infection

Objectives: Prevention of COVID-19, Lack of COVID-19 symptoms, and assessment of safety and tolerability

Procedure: Screening Period (Days −7 to −1): Prescription of home health monitoring equipment that includes a thermometer, a pregnancy test if applicable, and a daily diary. There are two groups being studied: Arm 1 and Arm 2.

Arm 1 is prescribed the following antimicrobials: hydroxychloroquine 200 mg twice a day for 1 day only, vitamin C 3000 IU per day for 12 weeks, vitamin D 3000 IU per day for 12 weeks, and zinc 50 mg per day for 12 weeks. The zinc can be reduced to 25 mg if GI upset occurs. The hydroxychloroquine is to be taken first thing in the morning as soon as subject has eaten and again right before bed, and must be separated from vitamin dose by at least 2 hours.

Arm 2 is prescribed the following antimicrobials: Placebo twice a day for 1 day only, vitamin C 3000 IU per day for 12 weeks, vitamin D 3000 IU per day for 12 weeks, and zinc 50 mg per day for 12 weeks. The zinc can be reduced to 25 mg if GI upset occurs.

Day 1: Patient is called to teach them how to use the diary in the EDC, discuss the medication regimen, and answer any questions they may have. Patient takes pregnancy test if applicable, collects a temperature reading, completes their diary, and takes the prescribed treatment regimen. Table 37 outlines the prescribed treatment regimen for Day 1.

TABLE 37

| | | |
|---|---|---|
| Drug | AM Dose | PM Dose |

| | | | | |
|---|---|---|---|---|
| Hydroxychloroquine (or placebo) | 200 | mg | 200 | mg |
| Vitamin C | 1500 | mg | 1500 | mg |
| Vitamin D | 1500 | IU | 1500 | IU |
| Zinc | 25 | mg | 25 | mg |

Day 2: Patient collects a temperature reading, completes their diary, and is called on the phone for assessment of any adverse events or serious adverse events, assessment of any COVID-19 symptoms, the list of prior and concomitant medications is updated, any questions the patient has are answered, and the patient takes their prescribed treatment regimen. Table 38 outlines the prescribed treatment regimen for Day 2.

TABLE 38

| | | |
|---|---|---|
| Drug | AM Dose | PM Dose |

| | | | | |
|---|---|---|---|---|
| Vitamin C | 1500 | mg | 1500 | mg |
| Vitamin D | 1500 | IU | 1500 | IU |
| Zinc | 25 | mg | 25 | mg |

Days 3-10: Patient collects a temperature reading, completes their diary, and takes their prescribed treatment regimen. Table 8 outlines the prescribed treatment regimen for Days 3-10.

TABLE 8

| | | |
|---|---|---|
| Drug | AM Dose | PM Dose |

| | | | | |
|---|---|---|---|---|
| Vitamin C | 1500 | mg | 1500 | mg |
| Vitamin D | 1500 | IU | 1500 | IU |
| Zinc | 25 | mg | 25 | mg |

Day 14: The patient is called for instruction on how to collect a nasal swab and package for shipping, assessment COVID-19 symptoms, updating their list of prior and concomitant medications, and answering any questions they may have.

Week 3: The patient is called for assessment of any adverse events or serious adverse events, assessment of any COVID-19 symptoms, updating their list of prior and concomitant medications, and answering any questions they may have. The patient takes a temperature reading, completes their diary, and takes the prescribed treatment regimen.

Table 39 outlines the treatment regime for week 3.

TABLE 39

| | | |
|---|---|---|
| Drug | AM Dose | PM Dose |

| | | | | |
|---|---|---|---|---|
| Vitamin C | 1500 | mg | 1500 | mg |
| Vitamin D | 1500 | IU | 1500 | IU |
| Zinc | 25 | mg | 25 | mg |

Week 4: The patient is called for assessment of any adverse events or serious adverse events, assessment of any COVID-19 symptoms, updating their list of prior and concomitant medications, and answering any questions they may have. The patient takes a temperature reading, completes their diary, and takes the prescribed treatment regimen. The patient also collects a nasal swab.

Table 40 outlines the treatment regime for week 4.

TABLE 40

| | | |
|---|---|---|
| Drug | AM Dose | PM Dose |

| | | | | |
|---|---|---|---|---|
| Vitamin C | 1500 | mg | 1500 | mg |
| Vitamin D | 1500 | IU | 1500 | IU |
| Zinc | 25 | mg | 25 | mg |

Weeks 5-11: The patient is called weekly for assessment of any adverse events or serious adverse events, assessment of any COVID-19 symptoms, updating their list of prior and concomitant medications, and answering any questions they may have. The patient takes a weekly temperature reading, completes their diary, and takes the prescribed treatment regimen.

Table 41 outlines the treatment regime for weeks 5-11.

TABLE 41

| | | |
|---|---|---|
| Drug | AM Dose | PM Dose |

| | | | | |
|---|---|---|---|---|
| Vitamin C | 1500 | mg | 1500 | mg |
| Vitamin D | 1500 | IU | 1500 | IU |
| Zinc | 25 | mg | 25 | mg |

Week 12: The patient presents to the clinic (or video conference) for evaluation that includes assessment for adverse events and serious adverse events, updating their list of prior and concomitant medications, a physical exam, nasal swab collection and COVID-19 sample collection.

Samples for COVID-19 testing are collected using synthetic swabs with plastic shafts. Nasal swabs are collected and immediately placed into a sterile vial with 2-3 mL of viral transport media. The vials are placed into biohazard bags, boxed up, the box sterilized, and picked up for shipment to the central laboratory. Samples are tested by RT-PCR.

Table 42 presents the schedule of events for Example 7.

TABLE 42

| | Screening | | | | | | | | |
|---|---|---|---|---|---|---|---|---|---|
| | (Day −7 | | | | | | | | |
| Assessment | to Day 1) | Day 1 | Day 2 | Day 3-10 | Day 14 | Week 3 | Week 4/Month 1 | Week 5 | Week 6 |
| Informed Consent & | X | | | | | | | | |
| Demographics | | | | | | | | | |
| Initial Review of | X | | | | | | | | |
| Prior and Concomitant | | | | | | | | | |
| Medications | | | | | | | | | |
| Medical Records | X | | | | | | | | |
| review, including | | | | | | | | | |
| baseline EKG | | | | | | | | | |
| Randomization | X | | | | | | | | |
| Prescription of | X | | | | | | | | |
| Hydroxychloroquine | | | | | | | | | |
| or placebo, vitamin C, | | | | | | | | | |
| vitamin D, and zinc | | | | | | | | | |
| Provision of daily | X | | | | | | | | |
| diary, thermometer | | | | | | | | | |
| and pregnancy test | | | | | | | | | |
| Pregnancy Test if | | X | | | | | | | |
| applicable | | | | | | | | | |
| Temperature at home | | X | X | X | X | X | X | X | X |
| Complete daily diary | | X | X | X | X | X | X | X | X |
| Hydroxychloroquine | | X | | | | | | | |
| 200 mg | | | | | | | | | |
| Vitamin C 3000 mg | | X | X | X | X | X | X | X | X |
| Vitamin D 3000 IU | | X | X | X | X | X | X | X | X |
| Zinc 50 mg^{a} | | X | X | X | X | X | X | X | X |
| Phone/video call | X | X | X | | X | X | X | X | X |
| to patient^{b} | | | | | | | | | |
| Update list of prior | | X | X | X | X | X | X | X | X |
| and concomitant | | | | | | | | | |
| medications | | | | | | | | | |
| Ask about AE and SAE | | X | X | X | X | X | X | X | X |
| Assessment of | | X | X | X | X | X | X | X | X |
| COVID-19 symptoms | | | | | | | | | |
| Swabs for RT-PCR | | X | | | X | | X | | |
| Vitals in-clinic^{c} | | | | | | | | | |
| Physical Exam | | | | | | | | | |

| Assessment | Week 7 | Week 8/Month 2 | Week 9 | Week 10 | Week 11 | Week 12/Month 3 |
|---|---|---|---|---|---|---|
| Informed Consent & | | | | | | |
| Demographics | | | | | | |
| Initial Review of | | | | | | |
| Prior and Concomitant | | | | | | |
| Medications | | | | | | |
| Medical Records | | | | | | |
| review, including | | | | | | |
| baseline EKG | | | | | | |
| Randomization | | | | | | |
| Prescription of | | | | | | |
| Hydroxychloroquine | | | | | | |
| or placebo, vitamin C, | | | | | | |
| vitamin D, and zinc | | | | | | |
| Provision of daily | | | | | | |
| diary, thermometer | | | | | | |
| and pregnancy test | | | | | | |
| Pregnancy Test if | | | | | | |
| applicable | | | | | | |
| Temperature at home | X | X | X | X | X | X |
| Complete daily diary | X | X | X | X | X | X |
| Hydroxychloroquine | | | | | | |
| 200 mg | | | | | | |
| Vitamin C 3000 mg | X | X | X | X | X | X |
| Vitamin D 3000 IU | X | X | X | X | X | X |
| Zinc 50 mg^{a} | X | X | X | X | X | X |
| Phone/video call | X | X | X | X | X | |
| to patient^{b} | | | | | | |
| Update list of prior | X | X | X | X | X | X |
| and concomitant | | | | | | |
| medications | | | | | | |
| Ask about AE and SAE | X | X | X | X | X | X |
| Assessment of | X | X | X | X | X | X |
| COVID-19 symptoms | | | | | | |
| Swabs for RT-PCR | | | | | | X |
| Vitals in-clinic^{c} | | | | | | X |
| Physical Exam | | | | | | X |

Regarding Table 42: The Zinc may be reduced to 25 mg if GI upset occurs, phone/video calls to the patient occur weekly during Weeks 2-11, and vitals in-clinic include height, weight, blood pressure (following 5 minutes sitting) pulse, respiratory rate, temperature, and oxygen saturation.

Statistical Analysis: The treated patients in this study are compared to the placebo group. Measurements include PCR test results, presence or absence of symptoms, and symptom severity.

The change of these measurements from the end to the baseline (post-pre) are used as the primary outcome, for example, μe=μe1−μe0, where μe1 and μe0 are the outcome of patients from the treatment group at the end and at baseline, respectively.

H₀:Δ≤δ₀ against H_{α}:Δ>δ₀ where δ0 is a clinically meaningful threshold to measure the disease symptoms. In this study, that meaningful threshold is calculated as mean change in clinical symptoms as recorded in the diary, from baseline through week 12. Each category in the diary is assigned a number, 0 for None, 1 for Mild, 2 for Moderate and 3 for severe. Each category is analyzed independently and as a group.

Categorical variables are summarized by presenting the number (n) and percent (%) of subjects in each category. All Statistical tests for the analysis are performed using the p<0.05 level of significance. All confidence intervals are one-sided.

Since these are healthcare workers who are exposed to COVID-19 at every shift, efficacy is determined by RT-PCR testing, as well as the presence or absence of symptoms as recorded in the patient diary via EDC.

### Example 8: Study of Microbiome of Patients with COVID Versus Patients without COVID

36 Covid-19 patients were studied in a cross-sectional study with 14 healthy controls (HC) to identify microbiome diversity by whole genome enrichment NGS. Patient records were included and compared any significant changes on species, genus, family, order, class, and phylum levels of the Covid-19 positive and HC population.

Deep shotgun microbiome sequencing analysis was performed on fecal samples from the 36 Covid-19 positive patients by whole genome enrichment NGS. The observed versus expected rates were primarily reported.

All individuals aged 3 years and older were eligible for inclusion. For each patient, a so-called period of eligibility for study inclusion was defined, which commenced on the latest of the study start date; A patient's period of eligibility ended on the earliest of registration termination; the end of data collection from their practice; or death.

Data Analysis

The differential taxa was conducted between the Gastrointestinal Microbiome of Covid-19 positive and HC relative abundance utilizing One Codex's bioinformatics analysis pipeline. For evaluating any statistical significance of the patient's data at each Classification at phylum, class, order, family, and genus levels, a Fisher's exact test was conducted between the two variables. Statistical analysis was conducted using chi-squared statistics by R version 3.6.1 (2019 Jul. 5). In the statistical analysis, p-value, Confidence interval and Odds ratio were considered for all comparisons.

Results

The study population included 50 patients (36 Covid-19 positives and 14 HC). For the HC population, data from patients after December 2019 were excluded from the study to avoid overlap between any Covid-19 possible positive patients.

The results from the 50 total patients that had their stool samples tested by whole genome enrichment NGS was evaluated. Detailed demographic and summary data, clinical characteristics including total numbers of diagnoses and events were analyzed for the study cohorts and are included in Table 43.

TABLE 43

| | | | Significant | | | Stool | Sx |
|---|---|---|---|---|---|---|---|
| | | | Past Medical | PCR | Covid -19 | Collection | Severity |
| No | Age | Sex | History | Date | PCR Results | Date | at BL |
| 1 | 20 | Female | Hypothyroid, | Jul. 10, 2020 | Positive | Jun. 24, 2020 | Severe |
| | | | Hashimoto, LCH | | | | |
| 2 | 21 | Female | Nothing to | No info | Positive | Apr. 5, 2020 | Moderate |
| | | | report | | | | |
| 3 | 56 | Male | Nothing to | No info | Positive | Apr. 5, 2020 | No info |
| | | | report | | | | |
| 4 | 25 | Female | Asthma, Reflux, | No info | Positive | Mar. 29, 2020 | Mild |
| | | | GI issue | | | | |
| 5 | 25 | Female | No info | No info | Positive | No info | Moderate |
| 6 | 44 | Female | Food | Apr. 8, 2020 | Positive | Apr. 13, 2020 | Severe |
| | | | sensitivities, gut | | | | |
| | | | issues | | | | |
| 7 | | No | No info | No info | Positive | No info | No info |
| | | info | | | | | |
| 8 | 53 | Male | No info | No info | Positive | No info | Severe |
| 9 | 23 | Male | No info | No info | Positive | No info | No info |
| 10 | 19 | Male | Nothing to | Apr. 22, 2020 | Positive | May 6, 2020 | No info |
| | | | report | | | | |
| 11 | 35 | Female | Seizure | No info | Positive | Jun. 23, 2020 | No info |
| 12 | 32 | Male | Nothing to | | Positive | Jun. 26, 2020 | Mild |
| | | | report | | | | |
| 13 | 63 | Female | No info | No info | Positive | Jul. 20, 2020 | No info |
| 14 | No | Male | No info | No info | Positive | Jun. 28, 2020 | No info |
| | info | | | | | | |
| 15 | 61 | Male | Nothing to | No info | Positive | No info | No info |
| | | | report | | | | |
| 16 | 50+ | Male | No info | No info | Positive | No info | No info |
| 17 | 48 | Female | No info | No info | Positive | No info | Severe |
| 18 | No | Female | No info | No info | Positive | Jul. 27, 2020 | No info |
| | info | | | | | | |
| 19 | 70 | Male | No info | No info | Positive | Aug. 5, 2020 | Severe |
| 20 | 56 | Male | Nothing to | No info | Positive | Aug. 10, 2020 | Severe |
| | | | report | | | | |
| 21 | 50+ | Male | No info | No info | Positive | No info | Moderate |
| 22 | 59 | Female | No info | No info | Positive | Sep. 4, 2020 | Severe |
| 23 | 35 | Female | No info | No info | Positive | No info | Moderate |
| 24 | 71 | Male | No info | No info | Positive | No info | No info |
| 25 | 58 | Female | No info | No info | Positive | No info | Moderate |
| 26 | 61 | Female | No info | No info | Positive | No info | No info |
| 27 | 55 | Male | Nothing to | | Positive | Oct. 21, 2020 | Severe |
| | | | report | | | | |
| 28 | 66 | Female | No info | No info | Positive | No info | Moderate |
| 29 | No | No | No info | No info | Positive | No info | No info |
| | info | info | | | | | |
| 30 | 66 | Female | No info | No info | Positive | No info | Severe |
| 31 | No | No | No info | No info | Positive | No info | No info |
| | info | info | | | | | |
| 32 | 61 | Female | No info | No info | Positive | No info | Severe |
| 33 | 69 | Male | No info | No info | Positive | No info | No info |
| 34 | No | No | No info | No info | Positive | No info | No info |
| | info | info | | | | | |
| 35 | 40 | Female | No info | No info | Positive | No info | Moderate |
| 36 | No | No | No info | No info | Positive | No info | No info |
| | info | info | | | | | |
| 37 | 6 | Male | Nothing to | Not | Not applicable- | No info | Not |
| | | | report | applicable | Negative | | applicable- |
| | | | | | | | Negative |
| 38 | 6 | Female | Nothing to | Not | Not applicable- | No info | Not |
| | | | report | applicable | Negative | | applicable- |
| | | | | | | | Negative |
| 39 | 8 | Female | Nothing to | Not | Not applicable- | No info | Not |
| | | | report | applicable | Negative | | applicable- |
| | | | | | | | Negative |
| 40 | 3 | Female | Nothing to | Not | Not applicable- | No info | Not |
| | | | report | applicable | Negative | | applicable- |
| | | | | | | | Negative |
| 41 | 11 | Male | Nothing to | Not | Not applicable- | No info | Not |
| | | | report | applicable | Negative | | applicable- |
| | | | | | | | Negative |
| 42 | 3 | Male | Nothing to | Not | Not applicable- | No info | Not |
| | | | report | applicable | Negative | | applicable- |
| | | | | | | | Negative |
| 43 | 4 | Female | Nothing to | Not | Not applicable- | No info | Not |
| | | | report | applicable | Negative | | applicable- |
| | | | | | | | Negative |
| 44 | 7 | Female | Nothing to | Not | Not applicable- | No info | Not |
| | | | report | applicable | Negative | | applicable- |
| | | | | | | | Negative |
| 45 | 16 | Female | Nothing to | Not | Not applicable- | No info | Not |
| | | | report | applicable | Negative | | applicable- |
| | | | | | | | Negative |
| 46 | 9 | Male | Nothing to | Not | Not applicable- | No info | Not |
| | | | report | applicable | Negative | | applicable- |
| | | | | | | | Negative |
| 47 | 10 | Female | Nothing to | Not | Not applicable- | No info | Not |
| | | | report | applicable | Negative | | applicable- |
| | | | | | | | Negative |
| 48 | 7 | Female | Nothing to | Not | Not applicable- | No info | Not |
| | | | report | applicable | Negative | | applicable- |
| | | | | | | | Negative |
| 49 | 55 | Female | Nothing to | Not | Not applicable- | No info | Not |
| | | | report | applicable | Negative | | applicable- |
| | | | | | | | Negative |
| 50 | 17 | Female | Nothing to | Not | Not applicable- | No info | Not |
| | | | report | applicable | Negative | | applicable- |
| | | | | | | | Negative |

Tables 44-49 demonstrate the association between relative abundance of the gastrointestinal microbiome at Phylum, Class, Order, Family and Species level of Covid-19 positive (Covid+) vs. healthy control (Covid−) patients. A 95% Confidence interval (CI) and odds ratio (OR) were used in addition to the p-value for better comparison.

TABLE 44

| | | | | | | Fisher exact test value |
|---|---|---|---|---|---|---|
| | | | | | Statistical | with 95% Confidence |
| Classification | Covid + Patients | | Covid − Patients | | Significance | Intervals(CI) and odds |
| Phylum | Presence | Absence | Presence | Absence | (P < 0.05) | ratio(OR) |
| Actinobacteria | 31 | 5 | 14 | 0 | Yes* | P < 0.00001(CI; |
| | | | | | | 0.00-0.3, OR = 0) |
| Firmicutes | 36 | 0 | 14 | 0 | No** | p-value 1 |
| Proteobacteria | 33 | 3 | 14 | 0 | Yes | P value 0.003(CI; |
| | | | | | | 0.00-0.48, OR = 0) |
| Bacteroidetes | 36 | 0 | 14 | 0 | No | p-value 1 |

TABLE 45

| | | | | | | Fisher exact test value |
|---|---|---|---|---|---|---|
| | | | | | Statistical | with 95% Confidence |
| Classification | Covid + Patients | | Covid − Patients | | Significance | Intervals(CI) and odds |
| Class | Presence | Absence | Presence | Absence | (P < 0.05) | ratio(OR) |
| Actinobacteria | 24 | 12 | 14 | 0 | Yes | P < 0.00001(CI; |
| | | | | | | 0.00-0.07, OR = 0) |
| Clostridia | 36 | 0 | 14 | 0 | No | p-value 1 |
| Negativicutes | 30 | 6 | 14 | 0 | Yes | P < 0.00001(CI; |
| | | | | | | 0.00-0.21, OR = 0) |
| Betaproteo- | 21 | 15 | 12 | 2 | Yes | P < 0.00001(CI; |
| bacteria | | | | | | 0.10-0.46, OR = 0.22) |
| Bacteroidia | 36 | 0 | 14 | 0 | No | p-value 1 |
| Gammaproteo- | 21 | 15 | 9 | 5 | No | p-value 0.46(CI; |
| bacteria | | | | | | 0.4-1.4, OR = 0.77) |
| Erysipelotrichia | 33 | 3 | 11 | 3 | Yes | p-value 0.009(CI; |
| | | | | | | 01.27-8.77, OR = 3.19) |

TABLE 46

| | | | | | | Fisher exact test value |
|---|---|---|---|---|---|---|
| | | | | | Statistical | with 95% Confidence |
| Classification | Covid + Patients | | Covid − Patients | | Significance | Intervals(CI) and odds |
| Order | Presence | Absence | Presence | Absence | (P < 0.05) | ratio(OR) |
| Bifidobacteriales | 20 | 16 | 14 | 0 | Yes | P < 0.00001(CI; |
| | | | | | | 0.00-0.05, OR = 0) |
| Clostridiales | 36 | 0 | 14 | 0 | No | p-value 1 |
| Veillonellales | 14 | 22 | 11 | 3 | Yes | P < 0.00001(CI; |
| | | | | | | 0.08-0.30, OR = 0.16) |
| Burkholderiales | 21 | 15 | 12 | 2 | Yes | P < 0.00001(CI; |
| | | | | | | 0.11-0.50, OR = 0.24) |
| Bacteroidales | 36 | 0 | 14 | 0 | No | p-value 1 |
| Erysipelotrichales | 33 | 3 | 14 | 0 | Yes | 0.003(CI; 0.00-0.48, |
| | | | | | | OR = 0) |
| Enterobacterales | 21 | 15 | 6 | 8 | Yes | p-value 0.03(CI; |
| | | | | | | 01.04-3.47, OR = 3.90) |

TABLE 47

| | | | | | | Fisher exact test value |
|---|---|---|---|---|---|---|
| | | | | | Statistical | with 95% Confidence |
| Classification | Covid + Patients | | Covid − Patients | | Significance | Intervals(CI) and odds |
| Family | Presence | Absence | Presence | Absence | (P < 0.05) | ratio(OR) |
| Bifidobacteriacae | 20 | 16 | 14 | 0 | Yes | P < 0.00001(CI; |
| | | | | | | 0.00-0.05, OR = 0) |
| Clostridiacae | 35 | 1 | 14 | 0 | No | p-value 0.24(CI; |
| | | | | | | 0.0-2.4, OR = 0) |
| Veilloneacellae | 15 | 22 | 11 | 3 | Yes | P < 0.00001(CI; |
| | | | | | | 0.05-0.25, OR = 0.12) |
| Ruminococcacea | 36 | 0 | 14 | 0 | No | p-value 1 |
| Sutterellacae | 19 | 17 | 11 | 3 | Yes | p-value 0.0003(CI; |
| | | | | | | 0.16-20.61, OR = 0.32) |
| Faecalibacterium | 31 | 5 | 14 | 0 | Yes | p-value 0.00007(CI; |
| | | | | | | 0.00-0.27, OR = 0) |
| Prevotellaceae | 17 | 19 | 10 | 4 | Yes | p-value 0.0009(CI; |
| | | | | | | 0.19-0.67, OR = 0.36) |
| Erysipelotrichaceae | 33 | 3 | 14 | 0 | Yes | p-value 0.006(CI; |
| | | | | | | 0.00-0.57, OR = 0) |
| Entero-bacteriaceae | 21 | 15 | 8 | 6 | No | p-value 1 |

TABLE 48

| | | | | | Statistical | |
|---|---|---|---|---|---|---|
| Classification | Covid + Patients | | Covid − Patients | | Significance | Fisher exact test |
| Genus | Presence | Absence | Presence | Absence | (P < 0.05) | value |
| Bifidobacterium | 20 | 16 | 14 | 0 | Yes | P < 0.00001(CI; |
| | | | | | | 0.00-0.05, OR = 0) |
| Clostridium | 33 | 3 | 14 | 0 | Yes | p-value 0.006(CI: |
| | | | | | | 0.00-0.57, OR = 0) |
| Veillonella | 14 | 22 | 11 | 3 | Yes | P < 0.00001(CI: |
| | | | | | | 0.08-0.30 0R = 0.16) |
| Ruminococcus | 30 | 6 | 14 | 0 | Yes | p-value 0.00007(CI; |
| | | | | | | 0.00-0.21, OR = 0) |
| Sutterella | 7 | 29 | 12 | 2 | Yes | P < 0.00001(CI; |
| | | | | | | 0.01-0.08 OR = 0.03) |
| Faecalibacterium | 31 | 5 | 14 | 0 | Yes | p-value 0.00007(CI; |
| | | | | | | 0.00-0.21, OR = 0) |
| Prevotella | 10 | 26 | 3 | 11 | No | p-value 0.40(CI; |
| | | | | | | 0.68-2.83, OR = 1.38) |
| Erysiplatoclostridium | 26 | 10 | 12 | 2 | Yes | p-value 0.03(CI; |
| | | | | | | 0.20-0.96, OR = 0.45) |
| Escherichia | 21 | 15 | 7 | 7 | No | p-value 0.32(CI; |
| | | | | | | 0.76-2.5, OR = 1.37) |
| Klebsiella | 3 | 33 | 1 | 13 | No | p-value 1 |

TABLE 49

| | | | | | Statistical | |
|---|---|---|---|---|---|---|
| Classification | Covid + Patients | | Covid − Patients | | Significance | Fisher exact test |
| Species | Presence | Absence | Presence | Absence | (P < 0.05) | value |
| Bifidobacterium | 20 | 16 | 14 | 0 | Yes | P < 0.00001(CI; |
| | | | | | | 0.00-0.05, OR = 0) |
| Clostridium | 33 | 3 | 14 | 0 | Yes | p-value 0.006(CI; |
| | | | | | | 0.00-0.57. OR = 0) |
| Veillonella | 14 | 22 | 11 | 3 | Yes | P < 0.00001(CI; |
| | | | | | | 0.08-0.30 0R = 0.16) |
| Ruminococcus | 30 | 6 | 14 | 0 | Yes | p-value 0.00007(CI; |
| | | | | | | 0.00-0.21, OR = 0) |
| Sutterella | 7 | 29 | 12 | 2 | Yes | P < 0.00001(CI; |
| | | | | | | 0.01-0.08 OR = 0.03) |
| Prevotella | 10 | 26 | 3 | 11 | No | p-value 0.40(CI; |
| | | | | | | 0.68-2.83, OR = 1.38) |
| Erysiplatoclostridium | 26 | 10 | 12 | 2 | Yes | p-value 0.03(CI; |
| | | | | | | 0.20-0.96, OR = 0.45) |
| Escherichia coli | 21 | 15 | 7 | 7 | No | p-value 0.32(CI; |
| | | | | | | 0.76-2.5, OR = 1.37) |
| Klebsiella | 3 | 33 | 1 | 13 | No | p-value 1 |

According to Tables 44-49, in the 50 patients tested in this study, the relative abundance of Actinobacteria P<0.00001 (CI; 0.00-0.3, OR=0) and Proteobacteria bacteria phyla p-value 0.003 (CI; 0.00-0.48, OR=0) were significantly less than HC. Although there was not any significant dysbiosis in both Bacteroidetes and Firmicutes phyla. At the class level, there was a significant reduction in Actinobacteria P<0.00001 (CI; 0.00-0.07, OR=0), Negativicutes P<0.00001 (CI; 0.00-0.21, OR=0), Betaproteobacteria P<0.00001 (CI; 0.10-0.46, OR=0.22) and Erysipelotrichia p-value 0.009 (CI; 01.27-8.77, OR=3.19).

At the order level, there were significant reductions in more bacteria, including, Bifidobacteriales P<0.00001 (CI; 0.00-0.05, OR=0), Veillonellales P<0.00001 (CI; 0.08-0.30, OR=0.16), Burkholderiales P<0.00001 (CI; 0.11-0.50, OR=0.24), and Erysipelotrichales 0.003 (CI; 0.00-0.48, OR=0) and significant increase in Enterobacterales p-value 0.03 (CI; 01.04-3.47, OR=3.90) compare to the HC.

At the family levels, Bifidobacteriacae P<0.00001 (CI; 0.00-0.05, OR=0), Veilloneacellae P<0.00001 (CI; 0.05-0.25, OR=0.12), Sutterellacae p-value 0.0003 (CI; 0.16-20.61, OR=0.32), Faecalibacterium p-value 0.00007 (CI; 0.00-0.27, OR=0), Prevotellaceae p-value 0.0009 (CI; 0.19- 0.67, OR=0.36) and Erysipelotrichaceae p-value 0.006 (CI; 0.00-0.57, OR=0) showed a significant reduction in Covid-19 patients compare to the HC.

At the genus level, significant reductions in relative abundances of Bifidobacterium P<0.00001 (CI; 0.00-0.05, OR=0), Clostridium p-value 0.006 (CI; 0.00-0.57, OR=0), Veillonella P<0.00001 (CI; 0.08-0.30 OR=0.16), Ruminococcus p-value 0.00007 (CI; 0.00-0.21, OR=0), Sutterella P<0.00001 (CI; 0.01-0.08 OR=0.03), Faecalibacterium p-value 0.00007 (CI; 0.00-0.21, OR=0) and Erysiplatoclostridium p-value 0.03 (CI; 0.20-0.96, OR=0.45) and Veillonella P<0.00001 (CI; 0.08-0.30 OR=0.16), were observed.

### Findings

Significant alterations of the fecal microbiota was observed, in some cases, for the first time, with special baseline characteristics in Covid-19 positive patients. Except for a significantly higher relative abundance of Enterobacterales at order level in Covid-19 positive patients, there was a significant relative abundance reduction in Bifidobacterium, Clostridium, Veillonella, Ruminococcus, Sutterella, Faecalibacterium, Erysiplatoclostridium, and Veillonella at the species and genus levels, Bifidobacteriacae, Veilloneacellae, Sutterellacae, Prevotellaceae and Erysipelotrichaceae at the family level, Bifidobacteriales, Veillonellales, Burkholderiales, and Erysipelotrichales at the order level, Actinobacteria, Negativicutes, Betaproteobacteria, and Erysipelotrichia class level and more importantly, Proteobacteria and Actinobacteria at the phylum level.

There were significant reductions in species, genus, family, order, class, and phylum level between Covid-19 positive patients and the control population. Dysbiosis was largest for Actinobacteria and Proteobacteria, at the phylum level, in the Covid-19 positive populations and must be addressed accordingly. The only significant abundance noted was at Enterobacterales order. At the class level, a significant reduction in Actinobacteria, Negativicutes & Betaproteobacteria was observed. At the order level, significant reductions in more bacteria, including, Bifidobacteriales, Veillonellales, Burkholderiales, and Erysipelotrichales, were observed. At the family levels, Bifidobacteriacae, Veilloneacellae, Sutterellacae, Faecalibacterium, Erysipelotrichales and Prevotellaceae showed a significant reduction in Covid-19 patients compared to the HC. At the genus level, significant reductions in relative abundances of Bifidobacterium, Clostridium, Veillonella, Ruminococcus, Sutterella, Faecalibacterium, and Erysiplatoclostridium were observed.

### Interpretation

Covid -19 positive patients will have a considerable reduction in microbiota composition and baseline microbiome dysbiosis, levels of cytokines, and inflammatory markers. Addressing the effects of microbial diversity and consequently, suppression of the immunologic response (known as the cytokine storm) is key to prevent and treat the disease. The importance of reduction at the phylum level must be considered as a possibility of an important indicator for Covid-19 positive susceptible patients. Application of diet-based improvement of microbiome would also be considered as an important preventive measure.

### Example 9: Bifidobacterium and Faecalibacterium Depletion, and Other Bacterial Composition Changes, as Potential Susceptibility Markers of SARS-Cov2 Severity

Objective: To compare gut microbiome diversity and composition in SARS-CoV-2 polymerase chain reaction (PCR)-confirmed positive patients whose symptoms ranged from severe to asymptomatic, and PCR-negative exposed healthy controls.

Summary of Design: Using a cross-sectional study design, shotgun next-generation sequencing (NGS) was sued to evaluate microbiome composition and diversity in patients with PCR-confirmed SARS-CoV-2 infections from March 2020 through January 2021 and SARS-CoV-2 PCR-negative exposed healthy controls. Patients were classified as being asymptomatic or having mild, moderate, or severe symptoms based on NIH criteria. Exposed healthy controls were individuals with prolonged or repeated close contact to patients with SARS-CoV-2 infection or their samples and consisted of family members of subjects or frontline healthcare worker. Microbiome diversity and composition were compared between patients and exposed-controls and across patient subgroups at all taxonomic levels.

Results: 52 patients and 20 controls were identified. Compared with controls, patients had significantly less bacterial diversity (p<0.05) and a lower relative abundance of Bifidobacterium, Faecalibacterium and other bacteria at the genus level. Additionally, there was an inverse association between disease severity and Bifidobacterium (P=0.001) relative abundance and bacterial diversity.

Conclusion: Depletion of Bifidobacteria spp. And Faecalibacterium either before or after infection minimized its immune-dampening effects and allowed SARS-Cov-2 infection to become symptomatic. Thus, low bacterial diversity and low Bifidobacteria abundance is a susceptibility signature for vulnerability to symptoms associated with SARS-CoV-2 infection.

Patients with SARS-CoV-2 infection possess significantly less bacterial diversity and lower abundance of Bifidobacteria and Faecalibacterium and increased abundance of Baceriodies at the genus level compared with exposed-healthy controls. There is an inverse association between disease severity and abundance of Bifidobacteria and Faecalibacterium and direct association of severity and Bacteriodes abundance.

### Additional Detail Regarding Study Design

Patients diagnosed with COVID-19 were recruited to the study following expression of interest or physician referral. Informed consent was obtained.

Patients aged ≥18 years of age, with RT-PCR-confirmed SARS-CoV-2 infection within one week of screening were eligible for enrolment. SARS-CoV-2 negative but exposed healthy controls included patients who were PCR-negative for SARS-CoV-2 and remained antibody-negative for between 3-6 months and asymptomatic for 6 months-1 year. SARS-CoV-2 negative exposed healthy controls were further required to meet the following criteria of they either shared a household with at least one symptomatic SARS-CoV-2-positive family member (e.g., sibling, parent, or child) or were a healthcare worker who had been repeatedly directly exposed to symptomatic SARS-CoV-2-positive patients for a minimum of six months or were exposed to numerous SARS-CoV-2-positive patient samples. Also, exposed healthy controls were those that, despite exposure to SARS-CoV-2, chose not to quarantine or take prophylaxis or treatment for COVID-19.

Patients undergoing treatment with total parenteral nutrition, or those with a history of significant gastrointestinal surgery (e.g. bariatric surgery, total colectomy with ileorectal anastomosis, proctocolectomy, postoperative stoma, ostomy, or ileoanal pouch) were excluded.

The study was conducted in accordance with ethical principles of the Declaration of Helsinki, the International Council for Harmonisation (ICH) Harmonised Tripartite Guideline for Good Clinical Practice (GCP), and the E and I Institutional Review Board (IRB) regulations. The clinical study protocol, a sample informed consent form, and other study-related documents were reviewed and approved by the Ethical and Independent Review. All patients provided written informed consent to participate in the study.

Symptomatic patients, as well as asymptomatic household contacts, whose SARS-CoV-2 infectivity was assessed by RT-PCR and exposed healthy controls were required to collect a stool sample. Patients were to collect one mL of fresh stool and place it directly in a Zymo Research DNA/RNA Shield fecal collection tube. Following fecal collection, individual subject DNA was extracted and purified with the Qiagen PowerFecal Pro DNA extraction kit. The isolated DNA was then quantitated utilizing the Quantus Fluorometer with the QuantFluor ONE dsDNA kit. After DNA quantification, the DNA was normalized, and libraries were prepared utilizing shotgun methodology with Illumina's Nextera Flex kit. Per the lab shotgun metagenomic processes, samples underwent tagmentation, amplification, indexing, and purification. After completing the NextSeq run, the raw data were streamed in real-time to Illumina's BaseSpace cloud for conversion to FASTQ files. The FASTQ data were compared to evaluate microbiome diversity in patients with PCR-confirmed SARS-CoV-2 infections and SARS-CoV-2 PCR-negative individuals. The COVID-19 patients had sought remote care in 2020 and 2021. The microbiome differences between the SARS-CoV-2 patients and SARS-CoV-2 exposed healthy controls were compared at all taxonomic levels.

The differences in relative abundance across taxa between the gut microbiome of SARS-CoV-2 infected patients and exposed healthy controls were assessed with One Codex's bioinformatics analysis pipeline using Jupyter notebook in Python. To evaluate the statistical significance of any variability noted between patients and exposed healthy controls at each taxonomic classification, ANOVA and t-test statistics were conducted by R version 3.6.1 (2019 Jul. 5), GraphPad, and SigmaPlot 12.0. Confidence intervals, and p-values were calculated for all comparisons in the statistical analysis, with P-values <0.05 considered significant. Shannon and Simpson indices were calculated by the bioinformatics software One Codex by Python based notebook software.

### Patient Characteristics

Demographic and clinical characteristics of patients (n=52) and exposed healthy controls (n=20) are presented in Table 19. Half (50%) of patients and 36% of exposed healthy controls were male. The median (SEM) age of patients was 53±2.54 years and of exposed healthy controls was 48±3.62 years. A total of 85% of patients were non-Hispanic White; 12.5% were Hispanic; and 2.5%, Black and 91% of exposed healthy controls were non-Hispanic White and 9% were Black. Nearly two-thirds of patients (62.5%) had underlying comorbidities considered risk factors for increased morbidity and mortality by CDC.1 Of the SARS-CoV-2 positive individuals, 57.7% had severe disease, 23.1% had moderate disease, and 11.5% had mild disease. The remaining 7.7% were asymptomatic. More than half (54.5%) of exposed controls had underlying comorbidities. The median (SEM) BMI of the 52 patients for whom data were available was 27.9+0.94 compared with 25.1+0.96 for the 20 exposed healthy controls.

Of the healthy exposed subjects, 16 were family members of one of the COVID-19 positive subjects, and 2 were Health Care workers with extensive, non-protected, exposure to subjects and 2 were laboratory personnel exposed to thousands of COVID-19 samples. Given the low number of health care worker subjects, statistics were not performed comparing similarity to family member groups. None of the patients or controls were vaccinated or on COVID-19 prophylaxis or treatment.

TABLE 50

| | | | | | | | PCR | |
|---|---|---|---|---|---|---|---|---|
| No | Age | Sex | Ethnicity | State | Risk Factors | BMI* | Result | Category |
| 1 | 21 | F | W | PA | none | 24.8 | positive | severe |
| 2 | 71 | M | H | CA | none | 18.4 | positive | severe |
| 3 | 86 | M | W | CA | Alzheimer's | 21.1 | positive | severe |
| 4 | 18 | F | W | AZ | none | 22 | positive | severe |
| 5 | 31 | M | W | CO | overweight | 26.9 | positive | severe |
| 6 | 35 | M | W | CA | obese | 30.7 | positive | severe |
| 7 | 43 | F | W | TX | overweight | 25.8 | positive | severe |
| 8 | 70 | F | W | LO | overweight | 25.8 | positive | severe |
| 9 | 56 | M | W | CA | overweight | 27.3 | positive | severe |
| 10 | 44 | F | W | GA | overweight | 27.8 | positive | severe |
| 11 | 53 | M | W | GA | obese | 30.7 | positive | severe |
| 12 | 37 | M | W | FL | hypertension | 23.8 | positive | mild |
| 13 | 60 | M | W | CO | none | 22.7 | positive | severe |
| 14 | 69 | F | W | CO | hypertension | 22.9 | positive | severe |
| 15 | 55 | M | W | NY | obesity, HIV | 37.2 | positive | severe |
| 16 | 67 | F | W | IL | hypertension, | 48.5 | positive | severe |
| | | | | | diabetes | | | |
| | | | | | mellitus, | | | |
| | | | | | obesity, | | | |
| | | | | | cerebral | | | |
| | | | | | aneurysm | | | |
| 17 | 66 | M | W | IL | hypertension, | 33.2 | positive | severe |
| | | | | | obesity, | | | |
| | | | | | pulmonary | | | |
| | | | | | embolus | | | |
| 18 | 49 | F | W | AL | overweight | 27.4 | positive | severe |
| 19 | 60 | M | W | CA | hypertension, | 26.2 | positive | severe |
| | | | | | overweight | | | |
| 20 | 63 | F | W | GA | hypertension, | 28.1 | positive | severe |
| | | | | | overweight | | | |
| 21 | 59 | F | W | GA | hypertension | NA | positive | severe |
| 22 | 53 | F | W | CA | none | 24.5 | positive | severe |
| 23 | 36 | M | W | OR | obese | 30.7 | positive | severe |
| 24 | 10 | M | W | HI | none | 14.5 | positive | severe |
| 25 | 52 | M | W | HI | none | 24.1 | positive | severe |
| 26 | 55 | F | W | HI | hypertension, | 25 | positive | severe |
| | | | | | overweight | | | |
| 27 | 61 | F | W | WA | none | 23.8 | positive | severe |
| 28 | 72 | F | W | CA | none | 19.5 | positive | severe |
| 29 | 62 | M | W | TN | obese, | 31.5 | positive | severe |
| | | | | | hypertension | | | |
| 30 | 18 | M | W | AZ | Crohn's | 15.1 | positive | severe |
| 31 | 23 | F | W | CA | obesity | 30.9 | positive | moderate |
| 32 | 25 | F | W | CA | obesity, asthma | 30.6 | positive | moderate |
| 33 | 24 | F | W | AZ | asthma | NA | positive | moderate |
| 34 | 36 | F | H | AZ | hypertension, | 25.4 | positive | moderate |
| | | | | | overweight | | | |
| 35 | 33 | M | H | CA | none | 18.4 | positive | moderate |
| 36 | 63 | F | H | CA | overweight | NA | positive | moderate |
| 37 | 65 | M | W | TX | COPD | NA | positive | moderate |
| 38 | 75 | M | W | CA | hypertension, | 48.5 | positive | moderate |
| | | | | | severe obesity | | | |
| 39 | 67 | M | W | CA | obesity | 33.1 | positive | moderate |
| 40 | 70 | M | W | GA | obesity | 30.7 | positive | moderate |
| 41 | 57 | F | W | TX | obesity | 32 | positive | moderate |
| 42 | 69 | F | W | NE | hypertension | 20.9 | positive | moderate |
| 43 | 19 | M | W | GA | none | 23.5 | positive | mild |
| 44 | 36 | F | W | NY | none | 18.6 | positive | mi |
| 45 | 35 | F | W | CA | overweight | 28.5 | positive | mild |
| 46 | 53 | M | W | NE | overweight | 29 | positive | mild |
| 47 | 38 | F | 1 | TN | obese | 31.3 | positive | mild |
| 48 | 45 | F | W | PA | hypertension | 23 | positive | mild |
| 49 | 22 | M | W | GA | overweight | 25.8 | positive | asymptomatic |
| 50 | 48 | F | H | CA | none | 20.4 | positive | asymptomatic |
| 51 | 67 | M | W | WI | overweight | 28.5 | positive | asymptomatic |
| 52 | 53 | M | W | IL | obese, | 35.3 | positive | asymptomatic |
| | | | | | diabetes | | | |
| 53 | 68 | F | W | WI | overweight | 26.6 | negative | n/a |
| 54 | 58 | F | H | PA | obesity, | 30 | negative | n/a |
| | | | | | exposed to + | | | |
| | | | | | daughter | | | |
| 55 | 51 | F | W | CA | hypertension, | 25 | negative | n/a |
| | | | | | prediabetes, | | | |
| | | | | | exposed to + | | | |
| | | | | | son, + | | | |
| | | | | | husband, and + | | | |
| | | | | | daughter | | | |
| 56 | 63 | M | W | CA | overweight | 26.4 | negative | n/a |
| | | | | | exposed to + | | | |
| | | | | | mother | | | |
| 57 | 29 | M | W | CA | overweight: | 26.4 | negative | n/a |
| | | | | | exposed to + | | | |
| | | | | | brother | | | |
| 58 | 23 | F | W | CA | overweight: | 26.4 | negative | n/a |
| | | | | | exposed to + | | | |
| | | | | | brother | | | |
| 59 | 62 | F | W | CA | none:exposed | 17.4 | negative | n/a |
| | | | | | to (kissed) + | | | |
| | | | | | boyfriend | | | |
| 60 | 51 | F | W | CA | none:exposed | 19.9 | negative | n/a |
| | | | | | to + son, + | | | |
| | | | | | husband, and + | | | |
| | | | | | daughter | | | |
| 61 | 16 | F | W | CA | none:exposed | 21.2 | negative | n/a |
| | | | | | to + mom | | | |
| 62 | 26 | M | B | CA | none:exposed | 19.5 | negative | n/a |
| | | | | | to + patients | | | |
| 63 | 56 | F | W | CA | overweight: | 25.5 | negative | n/a |
| | | | | | exposed to + | | | |
| | | | | | patient | | | |
| | | | | | samples | | | |
| 64 | 53 | F | W | CA | none:exposed | 24 | negative | n/a |
| | | | | | to workers, and + | | | |
| | | | | | patients and + | | | |
| | | | | | patient | | | |
| | | | | | samples | | | |
| 65 | 35 | M | W | KS | none:exposed | 23.7 | negative | n/a |
| | | | | | to + wife | | | |
| 66 | 35 | M | W | CA | none:exposed | 21.7 | negative | n/a |
| | | | | | to + roommate | | | |
| 67 | 64 | F | H | TN | prediabetic, | 26 | negative | n/a |
| | | | | | overweight | | | |
| 68 | 43 | F | W | CA | overweight, | 26.6 | negative | n/a |
| | | | | | exposed to + | | | |
| | | | | | patient | | | |
| | | | | | samples | | | |
| 69 | 45 | F | W | CA | obese, exposed | 35.1 | negative | n/a |
| | | | | | to + patients | | | |
| 70 | 44 | M | W | CA | overweight, | 27.2 | negative | n/a |
| | | | | | exposed to + | | | |
| | | | | | patients and + | | | |
| | | | | | patient | | | |
| | | | | | samples | | | |
| 71 | 52 | M | W | CA | overweight, | 27.1 | negative | n/a |
| | | | | | exposed to + | | | |
| | | | | | patients | | | |
| 72 | 15 | F | W | AZ | none: exposed | 16.9 | negative | n/a |
| | | | | | to + brother | | | |

*BMI, body mass index.

### Gut Microbiome Diversity and Composition

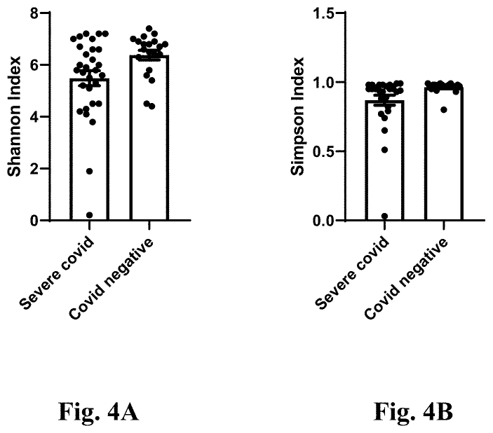

Alpha diversity in patients and exposed healthy controls are shown in FIG. 4A (Difference in Shannon Index for genus and family levels [95% CI 0.11 to 1.67, P=0.026]) and FIG. 4B (Difference in Simpson Index [95% CI 0.0031 to 0.18, P=0.043]) suggesting that the gut microbiome of patients was less diverse than that of exposed healthy controls.

Microbiome analysis revealed significant differences between the two groups. Table 51 lists the bacteria that had significant or otherwise different abundance of bacteria, either comparing difference due to COVID-19 severity (center column) among COVID-19 positive subjects or difference due to COVID-19 positivity (right column) among all subjects. Patients with PCR-confirmed SARS-CoV-2 infection showed a significantly decreased abundance of Bifidobacterium spp., Clostridium, Faecali NR, Faecalibacterium prausnitzii, Ruminococcus, and Subdolingranulum and increased abundance of Bacteriodes. (Table 51 right column). Table 51 left column shows the abundance of bacteria was affected by patient's level of symptom severity. A one-way ANOVA analysis revealed a significant relation between increased disease severity and decrease in abundance of Bifidobacterium spp., Faecali NR, Faecalibacterium prausnitzii, Ruminococcus, and Subdolingranulum and increase in abundance of Bacteriodes and Dorea.

TABLE 51

| | P-value | P-value |
|---|---|---|
| | Does severity of | Does COVID-19 |
| | COVID-19 affect | positivity affect |
| | bacterial | bacterial |
| | abundance? | abundance? |
| Akkermansia | 0.0790 | 0.6933 |
| Alistpies | 0.7860 | 0.5922 |
| Bacteroides | 0.0380 ↑ | 0.0023 ↑ |
| Bifido | 0.0010 ↓ | 0.0001 ↓ |
| Blautia | 0.0800 | 0.2859 |
| Clostridium | 0.8420 | 0.0326 ↓ |
| Collinsella | 0.8080 | 0.9551 |
| Coprococcus | 0.2340 | 0.0607 |
| Dorea | 0.0380 ↑ | 0.3731 |
| Eubacterium | 0.7580 | 0.1825 |
| Faecalibacterium | 0.0100 ↓ | 0.0001 ↓ |
| Faecalibacterium prausnitzii | 0.0070 ↓ | 0.0060 ↓ |
| Klebsiella | 0.8780 | 0.6280 |
| Oscillobacter | 0.8830 | 0.6365 |
| Parabacteroides | 0.4070 | 0.4734 |
| Prevotella | 0.6770 | 0.9510 |
| Roseburia | 0.4030 | 0.1557 |
| Ruminococcus | 0.0900 | 0.0415 ↓ |
| Subdoligranulum | 0.8970 | 0.0058 ↓ |

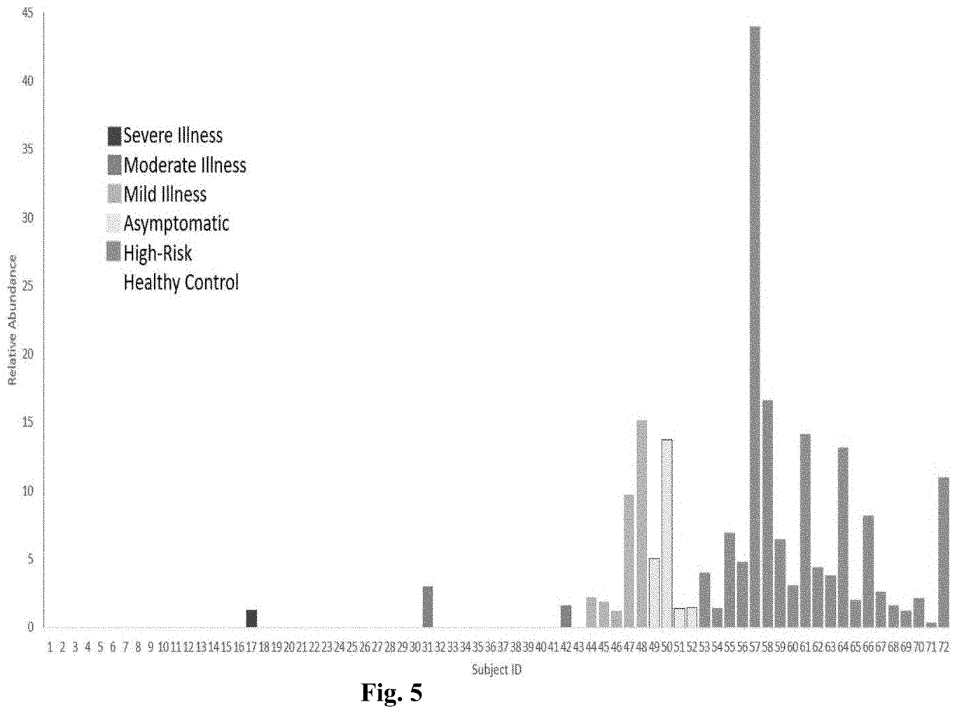

FIG. 5 shows the relative abundance of Bifidobacteria for each subject, grouped by COVID-19 severity. Analyzed via one-way ANOVA with Dunnett's post-hoc (comparing all COVID-19 severities including asymptomatic to healthy controls), there was a significant (P=0.0002) association between severity and Bifidobacteria relative abundance, particularly for severe (P<0.0001) and moderate severity (P=0.0031).

### Discussion

Immune function and health could be enhanced by bacterial abundance. Interactions between the host and gut microbiota are complex, numerous, and bidirectional. Gut microbiota regulate the development and function of the innate and adaptive immune system, potentially allowing them to protect against COVID-19. The study's main findings are that COVID-19 severity and positivity correlate to decreased levels of the pro-immune Bifidobacteria and Faecalibacteria and decreased levels of bacterial diversity. This was the first one to compare exposed to SARS-CoV-2 healthy subjects with COVID-19 affected subjects. That is, the following parameters were controlled: COVID-19 exposure, and observing bacterial changes are the associated with development of COVID-19 positivity.

Bacterial diversity is known to inversely relate to presence of various common disorders. This study showed that bacterial diversity indices (Shannon and Simpson Index), show that higher diversity correlates (p<0.05) to less COVID-19 severity, just as it reflects less vulnerability to many other disorders.

The genus Bifidobacterium has important immune functions, is a major component of the microbiome, and is frequently used in probiotics. Bifidobacteria are gram positive non-motile anaerobic rod-shaped bacteria. Bifidobacteria protect against intestinal epithelial cell damage and this protection is independent of their effects on tumor necrosis factor alpha (TNF-α) production. The exopolysaccharide coat which is a feature of some Bifidobacteria has been shown to play a significant role in this protective effect. Bifidobacteria also reduce cell damage by inhibiting TNF-α and macrophages. Also, Bifidobacteria increase Treg responses. These immune functions of Bifidobacteria could be critical for its COVID-protective effect.

Faecalibacterium genus and Faecalibacterium prausnitzii species levels were also inversely related to COVID-19 severity in this analysis. Age and diabetes are risk factors for COVID-19, and F. prausnitzii levels decline markedly in elder and diabetic populations. In fact, Faecalibacterium is considered an “indicator” of human health. The “western” diet (consumption of more meat, animal fat, sugar, processed foods, and low fiber) reduces the level of F. prausnitzii, while a high-fiber (e.g. Mediterranean diet of vegetables and fruits) and low meat diet enhances the count of F. prausnitzii. Preliminary studies show that reduced use of this Mediterranean diet within the same country was associated with increased COVID-19 death. In conclusion, it was shown that F. prausnitzii levels correlate to COVID severity and prior studies show that reduced F. prausnitzii is associated with COVID-19 vulnerabilities including age, diabetes, obesity, and possibly diet.

Evidence has accumulated to support a beneficial effect from supplementation with Bifidobacteria in a number of disease states. The number of commensal Bifidobacteria has been shown to decrease with age, a major COVID-19 risk factor. This study found that there is significant relationship between disease severity and relative abundance of Bifidobacterium spp.; patients with a more severe course of viral infection having decreased Bifidobacteria levels. However, it should be noted that there are no definitive studies concerning what constitutes a normal baseline abundance of Bifidobacteria in a “healthy” individual.

Changes in other bacteria were observed, characterized, namely: genus Bifidobacterium, Clostridium, Faecalibacterium (especially species F. prausnitzii), Subdolingranulum, and Ruminococcus showed decreased in bacterial abundance due to COVID-19 positivity, while genus Bacteroides showed increased bacterial abundance. Similarly, Bifidobacterium, Clostridium, and Faecalibacterium showed decreased bacterial abundance with increased COVID-19 severity, while Dorea and Bacteroides showed increased abundance.

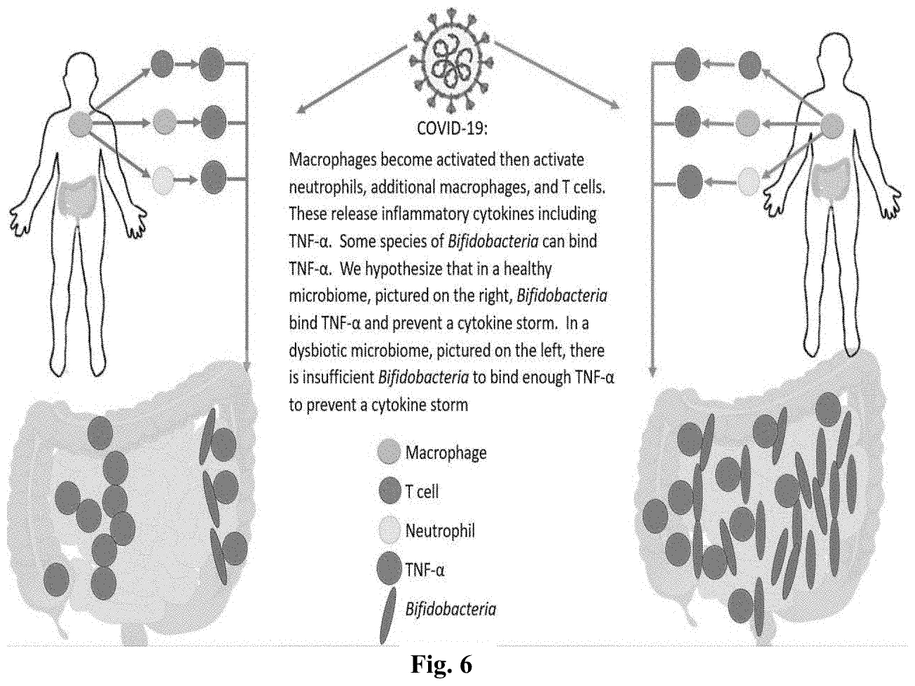

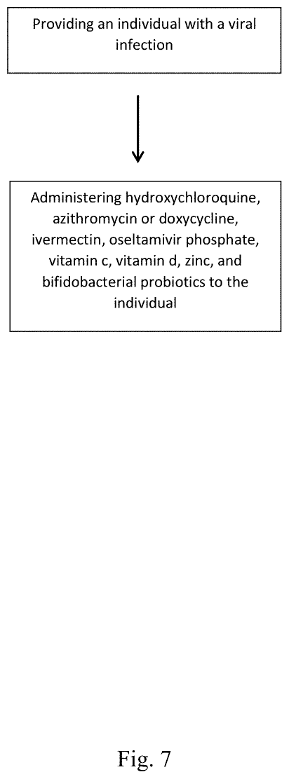

Innate immunity could be enhanced by increased bacterial level. COVID-19 pathology impacts include both direct effects from viral invasion and complex immunological responses including, in its most severe form, the so-called ‘cytokine storm.’ (FIG. 6) The cytokine storm is based upon innate immunity which is also enhanced by Bifidobacteria, Faecalibacteria, and bacterial diversity. Steroid treatment has situational success in COVID-19, and it is based on controlling an overactive innate immunity.

FIG. 8 shows the proposed mechanism for cytokine storm and immune hyper-response in SARS-CoV-2 positive patients. In individuals infected with SARS-CoV-2, the macrophages become activated; these in turn activate T-cells, additional macrophages, and neutrophils—all of which release cytokines, including TNF-α. Bifidobacteria, when present in sufficient numbers, can bind to TNF-α and prevent the subsequent cytokine storm. Therefore, patients with bifidobacterial dysbiosis lack this line of defense which may lead to a cytokine storm.

The findings reported in the current study support the role of commensal Bifidobacteria in the severity of viral infection, suggesting the importance of microbiota in viral pathogenesis and their potential for use in treatment (FIG. 8).

### Conclusions

Given the cross-sectional study design, it is not possible to determine whether the differences in levels of Bifidobacteria observed between patients and exposed healthy controls preceded or followed infection. If the differences preceded SARS-CoV-2 infection and are a marker of susceptibility, then boosting Bifidobacteria levels is be expected to decrease the risk or severity of SARS-CoV-2 infection. If changes followed SARS-CoV-2 infection, then adjunctive repopulation of the gut microbiome and boosting of Bifidobacteria through supplements or fecal transplant can speed recovery and reduce organ damage; particularly for patients hospitalized with ‘severe’ disease (including children with SARS-CoV-2-related multisystem inflammatory syndrome) and those with persistent symptoms.

COVID-19 ranges in presentation from asymptomatic to fatal, and bacterial composition and diversity also demonstrate marked variation. The same changes observed due to COVID-19, namely drop in Bifidobacterium and/or Faecalibacterium, underly COVID-19 risk factors including obesity, old age, and diabetes. Thus, bacterial levels, especially for Bifidobacterium and/or Faecalibacterium may be markers that determine the response (asymptomatic, severe, fatal) to infection by the same SARS-COV2 virus.

In summary, in this study, findings from only COVID-exposed subjects, both healthy and COVID-19 positive, were presented, and show the level of various bacteria are significantly related to the severity of COVID-19 and SARS-CoV-2 positive patients possessed significantly less bacterial diversity. These findings suggest that Bifidobacterium and other probiotic supplementation or repopulation via fecal transplant may speed recovery and reduce organ damage, particularly for patients hospitalized with severe disease. The data also suggest that characterizing the microbiome may allow the ability to predict vulnerability and appropriately treat COVID-19 patients in a manner dependent on their microbiome.

Having thus described the invention, it should be apparent that numerous structural modifications and adaptations may be resorted to without departing from the scope and fair meaning of the instant invention as set forth herein above and described herein below by the claims.

While particular forms of the invention have been illustrated and described, it will also be apparent to those skilled in the art that various modifications can be made without departing from the spirit and scope of the invention.

Although the present invention has been described in considerable detail with reference to certain preferred embodiments, other embodiments are possible. The forgoing description is not intended to be exhaustive or to limit the invention to the precise form disclosed. Many modifications and variations are possible in light of the above teaching. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments/methods contained in this disclosure. All references cited herein are incorporated by reference. Insofar as the description above and the accompanying drawings disclose any additional subject matter that is not within the scope of the claims below, the inventions are not dedicated to the public and the right to file one or more applications to claim such additional inventions is reserved.

What is claimed is:

1. A method of treating an individual having a viral infection or long hauler syndrome, the method comprising the steps of:
a) providing an individual having a viral infection or long hauler syndrome; and
b) administering thirteen antimicrobials to the individual, wherein the antimicrobials comprise:
i) administering hydroxychloroquine in a daily dosage range of 20 mg to 2,000 mg;
ii) administering azithromycin or doxycycline in a daily dosage range of 250 mg to 500 mg;
iii) administering ivermectin in a daily dosage range of 3 mg to 200 mg;
iv) administering oseltamivir phosphate in a daily dosage range of 30 mg to 75 mg;
v) administering famotidine in a daily dosage range of 20 mg to 80 mg;
vi) administering vitamin C in a daily dosage range of 1,000 mg to 20,000 mg;
vii) administering vitamin D in a daily dosage range of 1,000 IU to 100,000 IU;
viii) administering zinc in a daily dosage range of 25 mg to 100 mg;
ix) administering aspirin in a daily dosage range of 81 mg to 500 mg;
x) administering bifidobacterial probiotics in a daily dosage range of 1 billion to 100,000 billion bifidobacteria;
xi) administering cyproheptadine in a daily dosage range of 4 mg to 8 mg;
xii) administering a budesonide inhaler in daily dosage range of one to two puffs; and
xiii) administering an iodine nasal spray in a daily dosage range of one spray in one nostril to two sprays in both nostrils.

2. The method of claim 1, wherein steps b) i) through b) iii) and b) xi) comprise:
a) administering 400 mg of hydroxychloroquine;
b) administering 250 mg of azithromycin or doxycycline;
c) administering 36 mg of ivermectin; and
d) administering 8 mg of cyproheptadine.

3. The method of claim 1, wherein the method is administered for at least one day.

4. The method of claim 1, wherein the method is administered for at least two days.

5. The method of claim 1, wherein the method is administered for fourteen days.

6. The method of claim 1, further comprising step c) after step b), monitoring the individual's condition over a pre-determined period of time to determine whether the individual no longer has the viral infection.

7. The method of claim 1, wherein the probiotic is administered anally, orally, or topically.

8. The method of claim 7, wherein the probiotic is administered anally in the form of a fecal transplant, colonoscopy, suppository, enema, or topical application.

9. A method of treating an individual having a viral infection or long hauler syndrome, the method comprising the steps of:
a) providing an individual having a viral infection; and
b) administering eight antimicrobials to the individual, wherein the antimicrobials comprise:
i) administering hydroxychloroquine in a daily dosage range of 20 mg to 2,000 mg;
ii) administering azithromycin or doxycycline in a daily dosage range of 250 mg to 500 mg;
iii) administering ivermectin in a daily dosage range of 3 mg to 200 mg;
iv) administering vitamin C in a daily dosage range of 1,000 mg to 20,000 mg;
v) administering vitamin D in a daily dosage range of 1,000 IU to 100,000 IU;
vi) administering zinc in a daily dosage range of 25 mg to 100 mg; and
vii) administering bifidobacterial probiotics in a daily dosage range of 1 billion to 100,000 billion bifidobacterial.

10. The method of claim 9, further comprising the steps of:
a) administering famotidine in a daily dosage range of 20 mg to 80 mg;
b) administering oseltamivir phosphate in a daily dosage range of 30 mg to 75 mg;
c) administering aspirin in a daily dosage range of 81 mg to 500 mg;
d) administering cyproheptadine in a daily dosage range of 4 mg to 8 mg;
e) administering a budesonide inhaler in daily dosage range of one to two puffs; and
f) administering an iodine nasal spray in a daily dosage range of one spray in one nostril to two sprays in both nostrils.

11. The method of claim 10, wherein:
a) administering 400 mg of hydroxychloroquine;
b) administering 250 mg of azithromycin or doxycycline;
c) administering 36 mg of ivermectin; and
d) administering 8 mg of cyproheptadine.

12. The method of claim 9, wherein the method is administered for at least one day.

13. The method of claim 9, wherein the method is administered for at least two days.

14. The method of claim 9, wherein the method is administered for fourteen days.

15. The method of claim 9, further comprising step c) after step b), monitoring the individual's condition over a pre-determined period of time to determine whether the individual no longer has the viral infection.

16. The method of claim 9, wherein the probiotic is administered anally, orally, or topically.

17. The method of claim 16, wherein the probiotic is administered anally in the form of a fecal transplant, colonoscopy, suppository, enema, or topical application.

18. A combination of two or more antivirals for treating a viral infection or long hauler syndrome, the combination comprising at least two of the following: hydroxychloroquine, azithromycin or doxycycline, ivermectin, oseltamivir phosphate, famotidine, vitamin c, vitamin d, zinc, aspirin, bifidobacterial probiotics, cyproheptadine, budesonide inhaler, and iodine nasal spray.

19. The combination of claim 18, wherein the combination comprises hydroxychloroquine, azithromycin or doxycycline, ivermectin, oseltamivir phosphate, vitamin c, vitamin d, zinc, and bifidobacterial probiotics.

20. The combination of claim 19, wherein the combination comprises:
a) 20 mg to 2,000 mg of hydroxychloroquine;
b) 250 mg to 500 mg of azithromycin or doxycycline;
c) 3 mg to 200 mg of ivermectin;
d) 30 mg to 75 mg of oseltamivir phosphate;
e) 1,000 mg to 20,000 mg of vitamin C;
f) 1,000 IU to 100,000 IU of vitamin D;
g) 25 mg to 100 mg of zinc; and
h) 1 billion to 100,000 billion bifidobacterial in the form of a probiotic.
